(19) United States
(12) Patent Application Publication
(10) Pub. No.: US 2023/0017801 A1
(43) Pub. Date: Jan. 19, 2023

(54) SYSTEMS AND METHODS FOR DEPLOYING AN IMPLANTABLE MEDICAL DEVICE

(71) Applicant: Boston Scientific Scimed, Inc., Maple Grove, MN (US)

(72) Inventors: Eric Michael Petersen, Maple Grove, MN (US); John M. Edgell, Plymouth, MN (US)

(21) Appl. No.: 17/824,448

(22) Filed: May 25, 2022

Related U.S. Application Data
(60) Provisional application No. 63/221,155, filed on Jul. 13, 2021.

(51) Int. Cl. A61F 2/24

(52) U.S. Cl. CPC A61F 2/2466; A61F 2/2445; A61F 2220/0016; A61F 2250/001; A61B 2017/0649

(57) ABSTRACT

Improvements to devices, systems, and methods for delivering and/or deploying an implantable medical device are described. An implantable medical device may include an annuloplasty ring for implantation on a valve of a patient. Systems and methods may be configured to present graphical user interfaces with device images to implement efficient and accurate implantation of the implantable medical device. The device images may be based on sensor information obtained via sensors associated with the implantable medical device, such as a camera device, a diagnostic imaging device, position sensors, and/or the like. In other aspects, systems and methods may determine optimized configurations for the implantable medical device based on device characteristics including, without limitation, a shape formed by components of the implantable medical device and/or component coordinate information. Systems and methods may operate to facilitate deployment of the implantable medical device to correspond with the optimized configuration. Other embodiments are described.

### CROSS-REFERENCE TO RELATED APPLICATIONS

This application claims the benefit of priority under 35 U.S.C. § 119 to U.S. Provisional Application Ser. No. 63/221,155, filed Jul. 13. 2021, the disclosure of which is incorporated herein by reference in its entirety for all purposes.

### FIELD

The present disclosure relates generally to improvements to medical devices and methods of use of medical devices, including methods and systems for the delivery and/or deployment of implantable medical devices, for example, for implantation in a heart valve for treatment of a cardiac insufficiency.

### BACKGROUND

Abnormalities of the heart, such as heart disease, may lead to incompetent cardiac structures that require medical treatment. Conventional techniques for addressing serious structural heart conditions have involved open heart surgical procedures to implant a corrective device within the heart in an attempt to restore native heart operation. However, such procedures are highly invasive and are associated with elevated morbidity and mortality risks

Less invasive approaches have been developed to address structural heart abnormalities. For example, a transcatheter procedure may be used to deliver a corrective device to a target site within the heart through the circulatory system, such as via the femoral vein. Once the corrective device has been positioned within or adjacent to a target site, adjustment and final implantation (for instance, affixing the device to heart tissue) may be performed. Accordingly, a surgeon typically modifies the position, orientation, or other characteristic of components of the corrective device to achieve an optimized fit for the patient anatomy. A typical corrective device, such as an annuloplasty ring for treating valve regurgitation, will have multiple interrelated components that may each require configuration and/or affixation during an implantation procedure. Accordingly, such procedures are highly complex because a change in one characteristic of a corrective device component may have multiple consequences on other aspects of the corrective device and the performance thereof. As a result, it is challenging for the surgeon to effectively evaluate each potential consequence of a change to a corrective device, particularly in a time frame required during a live operation. As a result, a surgeon using a conventional system may have difficulty efficiently determining the optimized placement of a corrective device within the heart of a patient to address a heart abnormality.

It is with considerations of these and other challenges in mind that improvements such as disclosed herein may be useful.

### SUMMARY

This summary of the disclosure is given to aid understanding, and one of skill in the art will understand that each of the various features of the disclosure may advantageously be used separately in some instances, or in combination with other features of the disclosure in other instances. No limitation as to the scope of the claimed subject matter is intended by either the inclusion or non-inclusion of elements, components, or the like in this summary.

In accordance with various features of the present disclosure, is a method of deploying an annuloplasty ring within a heart of a patient for a closed valve repair procedure. In some embodiments, the method may include positioning the annuloplasty ring adjacent to a valve within a heart of the patient, the annuloplasty ring may include a plurality of anchors, a plurality of collars, and at least one sensor configured to provide sensor information indicating one of a position or configuration of the annuloplasty ring, receiving, at a logic device, sensor information associated with the annuloplasty ring, determining configuration information of the annuloplasty ring within the heart of the patient, displaying, via the logic device, a device image corresponding to the annuloplasty ring based on the configuration information, receiving input, at the logic device, configuring the device image to correspond to the configuration information, determining at least one adjustment for the annuloplasty ring based on the device image, configuring the annuloplasty ring based on the at least one adjustment, and implanting the annuloplasty ring within the patient by threading the anchors into valve tissue.

In some embodiments of the method, the at least one sensor may include at least one of a camera imaging device or a diagnostic imaging device. In various embodiments of the method, the at least one sensor may include intravascular cardiac echography (ICE) catheter arranged within the annuloplasty ring.

In some embodiments of the method, the configuration information may include an anchor placement status of the plurality of anchors or adjustment of the plurality of collars.

In exemplary embodiments of the method, the device image may include a simulated image overlaid on an image of a valve portion of the heart of the patient.

In some embodiments of the method, the at least one adjustment may include at least one of placement of the annuloplasty ring, placement of at least one of the plurality of anchors, an adjustment value of at least one of the plurality of collars. In some embodiments of the method, the at least one adjustment may be based on a determination that placement of at least one of the plurality of anchors will not enter sufficiently anchor into the valve tissue.

In various embodiments of the method, the method may include, via the logic device, determining an optimized configuration of the annuloplasty ring based on at least one of a shape of a frame of the annuloplasty ring or coordinates of the anchors. In exemplary embodiments of the method, the method may include, via the logic device, displaying at least one adjustment indicator to indicate an adjustment of at least one of the annuloplasty ring or at least one of the plurality of anchors.

In some embodiments of the method, the configuration of the annuloplasty ring may include a configuration of the plurality of anchors and the plurality of collars.

In accordance with various features of the present disclosure, is an apparatus that may include a storage device and logic, at least a portion of the logic implemented in circuitry coupled to the storage device to implement an implantation process to deploy an implantable medical device within a patient via a closed surgical process. In some embodiments, the logic may be configured to receive sensor information from at least one sensor associated with the implantable medical device, the implantable medical device may include a plurality of components, determine position information and configuration information of the implantable medical device based on the sensor information, generate a device image corresponding to the implantable medical device based on the position information and the configuration information, and determine an optimized configuration based on at least one configuration characteristic of the plurality of components using at least one computational model, the at least one configuration characteristic may include one of a shape of the implantable medical device or component coordinates.

In some embodiments of the apparatus, the implantable medical device may include an annuloplasty ring.

In various embodiments of the apparatus, the at least one sensor may include at least one of a camera imaging device, a diagnostic imaging device, a position sensor, or an accelerometer sensor. In various embodiments of the apparatus, the at least one sensor may include intravascular cardiac echography (ICE) catheter arranged within the annuloplasty ring.

In exemplary embodiments of the apparatus, the position information may include a position of the implantable medical device in relation to a target site and the configuration information may include at least one configuration setting of at least one of the plurality of components.

In some embodiments of the apparatus, the logic may be operative to cause an adjustment of at least one of the plurality of components to conform the implantable medical device to the optimized configuration.

In various embodiments of the apparatus, the configuration information may include at least one of an anchor placement status of the plurality of anchors or an adjustment value of the plurality of collars. In exemplary embodiments of the apparatus, the device image may include a simulated image overlaid on an image of a valve portion of the heart of the patient.

In some embodiments of the apparatus, the at least one adjustment may include at least one of placement of the annuloplasty ring, placement of at least one of the plurality of anchors, adjustment of at least one of the plurality of collars. In various embodiments of the apparatus, the logic, responsive to executing the instructions, may operate to display at least one adjustment indicator to indicate an adjustment of at least one of the annuloplasty ring or at least one of the plurality of anchors. In some embodiments of the apparatus, the configuration of the annuloplasty ring may include a configuration of the plurality of anchors and the plurality of collars

In accordance with various features of the present disclosure, is a computer-implemented method to deploy an implantable medical device within a patient via a closed surgical process. In various embodiments the method may include, via a computing device operably coupled to the implantable medical device receiving sensor information from at least one sensor associated with the implantable medical device, the implantable medical device may include a plurality of components, determining position information and configuration information of the implantable medical device based on the sensor information, generating a device image corresponding to the implantable medical device based on the position information and the configuration information, and determining an optimized configuration based on at least one configuration characteristic of the plurality of components using at least one computational model, the at least one configuration characteristic may include one of a shape of the implantable medical device or component coordinates.

In some embodiments of the computer-implemented method, the implantable medical device may include an annuloplasty ring. In some embodiments of the computer-implemented method, the at least one sensor may include at least one of a camera imaging device, a diagnostic imaging device, a position sensor, or an accelerometer sensor. In various embodiments of the method, the at least one sensor may include intravascular cardiac echography (ICE) catheter arranged within the annuloplasty ring

In some embodiments of the computer-implemented method, the position information may include a position of the implantable medical device in relation to a target site and the configuration information comprising at least one configuration setting of at least one of the plurality of components.

In some embodiments of the computer-implemented method, the method may further include causing an adjustment of at least one of the plurality of components to conform the implantable medical device to the optimized configuration.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 depicts an example of a first operating environment in accordance with one or more features of the present disclosure.

FIG. 2 depicts an example of a second operating environment in accordance with one or more features of the present disclosure.

FIG. 3 is a perspective view of an example of an implantable medical device in accordance with one or more features of the present disclosure.

FIG. 4 is a perspective view of the implantable medical device of FIG. 3 delivered to a target site in accordance with one or more features of the present disclosure.

FIG. 5 is a perspective view of an example of a delivery/deployment system formed in accordance with one or more features of the present disclosure with an example of an implantable medical device in a collapsed configuration.

FIGS. 6A-6H depict illustrative graphical user interfaces for performing a surgical procedure for deploying an implantable medical device in accordance with one or more features of the present disclosure.

FIG. 7 illustrates a workflow in accordance with one or more features of the present disclosure.

FIG. 8 illustrates a logic flow in accordance with one or more features of the present disclosure.

FIG. 9 illustrates an embodiment of a computing architecture in accordance with one or more features of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably without intent to limit or otherwise), etc., such as when using a device (e.g., introducing the device into a patient, or during delivery, deployment, implantation, positioning, etc.), and “distal” refers to the direction or location furthest from the user, such as when using the device (e.g., introducing the device into a patient, or during delivery, deployment, implantation, positioning, etc.). “Longitudinal” means extending along the longer or larger dimension of an element. “Central” means at least generally bisecting a center point, and a “central axis” means, with respect to an opening, a line that at least generally bisects a center point of the opening, extending longitudinally along the length of the opening when the opening comprises, for example, a tubular element, a strut, a channel, or a bore.

In accordance with aspects of the present disclosure, a system for deploying and/or delivering an implantable medical device (referenced herein as a deployment system for the sake of simplicity and without intent to limit), as disclosed herein with reference to various embodiments, includes a deployment device structure configured to deliver the implantable medical device to a target site and/or manipulate the implantable medical device at the target site and a computer-assisted deployment system configured for use in planning and/or performing a surgical implantation procedure.

In various embodiments, the deployment device structure may include at least one flexible elongate member (such as in the form of a wire, shaft, etc.) capable of being navigated through a tortuous path (for instance, a patient circulatory system) within the human body and configured to engage a portion of the implantable device (for example, to deliver the implantable device to a treatment site, to deploy the implantable device, to implant the implantable device, to manipulate the implantable device, etc.).

In some embodiments, a computer-assisted deployment system may operate to provide a surgical planning process configured for use in a cardiac procedure, for example, for delivering, positioning, and/or implanting an implantable medical device within the heart of a patient. In one non-limiting example, the surgical process may include a heart valve repair surgery. For instance, a heart valve repair surgery may include implanting an annuloplasty ring on, around, or adjacent to a heart valve (for instance, a mitral valve or tricuspid valve) in an attempt to restore the valve annulus to a native configuration to address a heart condition, such as a valve regurgitation condition. In one non-limiting example, a surgical planning process may include a method of determining placement parameters or other information associated with implantation and/or configuration of the implantable medical device at the target site.

Although valve repair surgery and annuloplasty ring systems for implantation on or about a heart valve are used as examples in the present disclosure, embodiments are not so limited as these examples are provided for illustrative purposes. A person of skill in the art would recognize that the described embodiments may be applied to various other types of surgeries, implantable medical devices, anatomical regions, and/or the like.

A heart valve repair surgery may be required for a valve regurgitation condition arising due to expansion and/or weakening of the chambers of the heart. A valve regurgitation condition may occur when the annulus of a heart valve, such as the mitral valve, dilates excessively such that valve leaflets no longer effectively close during systolic contraction. As a result, regurgitation (for instance, retrograde flow back across the valve that should be closed) of blood occurs during ventricular contraction leading to reduced cardiac output.

Conventional techniques for addressing valve regurgitation have involved open heart surgical procedures to implant an annuloplasty ring within the heart in an attempt to restore the valve annulus to a native configuration. However, such procedures are highly invasive and are associated with high morbidity and mortality risks. In addition, patients with certain health conditions are not candidates for open heart surgery. Less invasive approaches have been developed to treat a valve regurgitation condition due to an insufficient heart valve. For example, a transcatheter procedure may be used to deliver an annuloplasty ring to a target site within the heart through the circulatory system, such as via the femoral vein.

Particular features for various embodiments of an implantable medical device, of a deployment device structure, a computer-assisted deployment system, and of related systems and methods of use of the foregoing (either together or separately), are described herein. In one non-limiting example, an implantable medical device may be the same or similar to the Millipede System (e.g., the Millipede Transcatheter Annuloplasty Ring System) provided by Boston Scientific Scimed, Maple Grove, Minn., United States of America. In other non-limiting examples, an implantable medical device and related systems and methods of use may have the same or similar features and/or functionalities as other implants, delivery systems, and related systems and methods of use as described, for example, without limitation, in U.S. Pat. No. 9,180,005, issued Nov. 10, 2015, and titled “Adjustable Endoluminal Valve Ring”; U.S. Pat. No. 10,335,275, issued Jul. 2, 2019, and titled “Methods for Delivery of Heart Valve Devices Using Intravascular Ultrasound Imaging”; U.S. Pat. No. 9,848,983, issued Dec. 26, 2017, and titled “Valve Replacement Using Rotational Anchors”; U.S. Pat. No. 10,555,813, issued Feb. 11, 2020, and titled “Implantable Device and Delivery System for Reshaping a Heart Valve Annulus”; U.S. Pat. No. 10,548,731, issued Feb. 4, 2020, and titled “Implantable Device and Delivery System for Reshaping a Heart Valve Annulus”; U.S. Pat. No. 9,192,471, issued Nov. 24, 2015, and titled “Device for Translumenal Reshaping of a Mitral Valve Annulus”; U.S. Patent Application Publication No. 2010/0249920, published Sep. 30, 2010, and titled “Device for Translumenal Reshaping of a Mitral Valve Annulus”; U.S. Pat. No. 9,795,480, issued Oct. 24, 2017, and titled “Reconfiguring Tissue Features of Heart Annulus”; U.S. Pat. No. 9,610,156, issued Apr. 4, 2017, and titled “Mitral Valve Inversion Prosthesis”; U.S. Pat. No. 10,321,999, issued Jun. 18, 2019, and titled “Systems and Methods for Reshaping a Heart Valve”; U.S. Provisional Patent Application No. 63/193,236, filed on May 26, 2021, and titled “Devices, Systems, and Methods for Implanting and Imaging Cardiac Devices”; and/or U.S. Provisional Patent Application No. 63/193,230, filed on May 26, 2021, and titled “Devices, Systems, and Methods for Implanting and Imaging Cardiac Devices”; each of which is incorporated herein by reference in its entirety for all purposes.

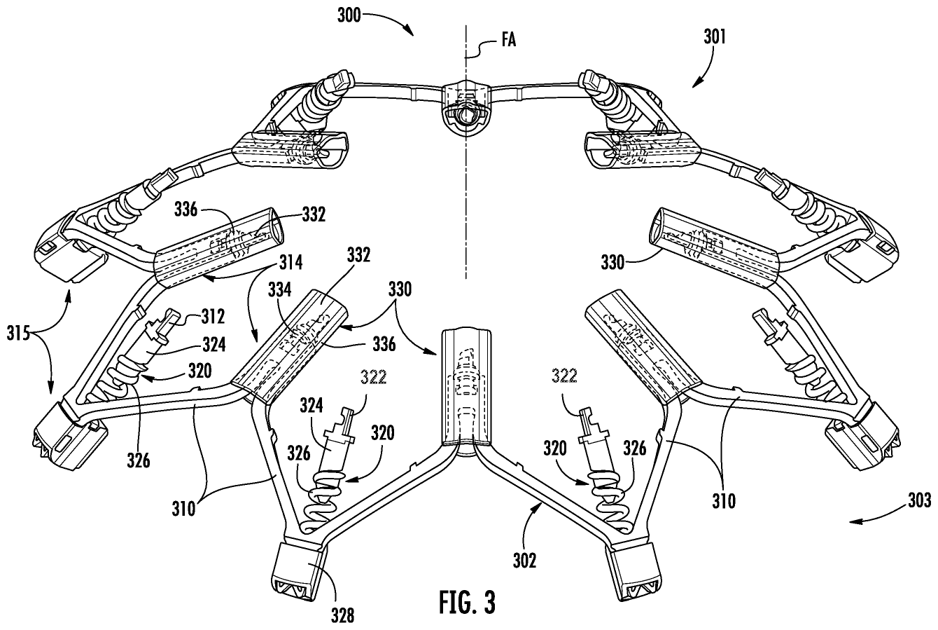

An implantable medical device may include various components that require correct placement and/or configuration at a target site (for instance, see anchors 320 and/or collars 330 of implantable device 300 of FIG. 3). For a non-invasive procedure, a surgeon may rely on diagnostic imaging techniques to view the implantable medical device and/or components thereof, such as ultrasound, echocardiography, computed tomography (CT), magnetic resonance imaging (MRI), and/or the like. With such systems, the implantable medical device is typically partially visible at best. For example, components on only one side of the implantable medical device may be visible at a time. In another example, only one perspective of the implantable medical device (for instance, an internal side view) may be available, while other perspectives (for instance, a top-down view) may not be visible. Accordingly, a surgeon may not have an adequate and comprehensive view of the implantable medical device and all of the associated components during a surgical procedure using conventional techniques and systems.

Surgical implantation processes are influenced by the anatomy of the patient and/or the particular condition of the patient. For example, the anchors of an annuloplasty ring may require affixation to different portions of a heart valve (for instance, a leaflet, an annulus, an atrium, and/or the like) of a first patient compared with a second patient. In another example, the collars of the annuloplasty ring may require different settings to achieve a different cinching configuration for a patient based on the patient's specific valve anatomy and condition.

For instance, in an annuloplasty ring example, it may be desirable to adjust the overall implant size and/or shape to adjust the size and/or shape of the valve in which the implantable device has been implanted. In some embodiments, the implantable device may be configured (such as with struts joined along distal and proximal apices) to be expandable or retractable (e.g., radially) to move or otherwise be adjusted between a reduced-diameter configuration for delivery and an expanded-diameter configuration for implantation. It may be desirable to further adjust the overall diameter or configuration of the implantable device once implanted, in order to adjust the shape of the valve, such as to effect improved closure thereof. Cinch devices may be provided on the implantable device and movable to adjust the relative positions of the anchors (generally, once the anchors have been implanted) to adjust the configuration of the implantable device and/or the implant location. For instance, in some embodiments the implantable device is implanted around a cardiac valve, such as the mitral valve or the tricuspid valve, with a plurality of spaced apart anchors. Adjustment of the relative positions of one or more anchors once implanted around the valve adjusts the shape of the valve to facilitate repair of the valve. In some embodiments, a cinch collar or collar or slider (such terms being used interchangeably herein without intent to limit) may be provided over components of the implantable device to draw together such components or to allow such components to move apart to affect the shape and configuration of the implantable device and, consequently, the shape and configuration of the tissue to which the implantable device is secured. For instance, in an implantable device formed with struts joined at distal and proximal apices, the collar may be positioned over the proximal apices and advanced or retracted to affect the relative orientations of adjacent struts to affect the configuration of the frame. An actuator or driver shaft (such as that used to implant the anchors) may be engaged with the implantable device, such as with the collar or an actuator thereof (e.g., a rotatable shaft advancing or retracting the collar upon being rotated by the actuator), to adjust the implantable device. The flexible elongate member and associated latch may be coupled to a coupler on the collar or the collar actuator. The actuator or driver shaft may be engaged over the latch and the coupler and rotate both to adjust the position of the collar to adjust the implantable device

In general, conventional implantation techniques involve the surgeon choosing the location and/or configuration of the implant via a “free placement” process where the surgeon is tasked with determining the optimized location and/or configuration of the implant via a manual process based on limited visual information. For example, in an annuloplasty ring implantation procedure, the surgeon may manually track (for instance, via a whiteboard, electronic document, and/or the like) the location and configuration of anchors and collars in one location, while viewing the implant in another location. Such “free placement” requires the surgeon to simultaneously evaluate a multitude of possible configurations, locations, parameters and or the like, which is time consuming and prone to error (particularly during a live surgical procedure). In addition, movement of one component (for instance, a first anchor or collar) may affect the position and/or configuration of one or more other components (for instance, a second and/or third anchor or collar), as well as modify the structure and/or performance of the valve. In addition, “free placement” allows the surgeon to change the implant position and/or configuration into sub-optimized arrangements. The numerous and intricate variables for implantable device positioning and/or configuring lead to a multidimensionally complex configuration space which the surgeon has to evaluate while trying to achieve the goals of the surgery, for instance, achievement of valve performance, monitoring of vitals, and/or the like.

Accordingly, some embodiments may include methods, systems, and/or apparatuses that provide computer-assisted surgical processes operative to visualize, emulate, or otherwise present implantable medical devices for positioning and/or implantation. In some embodiments, a computer-assisted surgical process may operate to present a device image configured to provide an accurate and comprehensive view of the implantable medical device to a surgeon during a surgical procedure. In various embodiments, the device image may be based on a mapping of the physical implantable medical device within the patient based on device information obtained via one or more sensors, for instance, a camera or a diagnostic imaging device. In some embodiments, the map may be or may include a three-dimensional (3D) image of the implantable medical device in association with the patient anatomy. In exemplary embodiments, the device image may provide a real-time or substantially real time mapping of the implantable medical device based on the device information.

Embodiments of the present disclosure provide numerous technological advantages and technical features over conventional systems. One non-limiting example of a technological advantage may include providing a surgical planning process using images and/or models of an implantable medical device within patient anatomy. An additional non-limiting example of a technological advantage may include emulating an annuloplasty ring and components thereof to present a surgeon with an accurate and comprehensive view of the annuloplasty ring during a surgical procedure. Surgical planning processes according to some embodiments may, among other things, facilitate planning of implantable medical device positioning with decreased complexity and improved efficiency and effectiveness as compared to conventional systems. A further non-limiting example of a technological advantage may include providing improved user interfaces and workflows for planning valve repair procedures, which have not been adequately addressed by conventional systems. A further non-limiting example of a technological advantage may include providing a computer-based surgical process that may be performed via a methodical approach that is more efficient and less error prone than conventional implantation techniques. Conventional computing devices have only been capable of simply providing bare images of an implant device to the surgeon. Embodiments are not limited in this context. Additional technological advantages and technical features over conventional systems would be known to those of skill in the art based on the present disclosure.

Embodiments described in the present disclosure may provide improvements in computing technology. For example, managing the complexity of the placement and implantation of an implantable medical device in or on patient anatomy has long been problematic for healthcare professionals using conventional computer-assisted tools, particularly managing changes during a surgical procedure. The placement of an implantable medical device, such as an annuloplasty ring, involves multiple parameters and other configurations (for example, anchor placement, collar position, and/or the like). These parameters lead to a multidimensionally complex configuration space which the surgeon (or other healthcare professional) has to explore while trying to achieve the goals of the surgery. A change in the implant position and/or parameters may have a cascading effect on multiple other properties, that may not be noticeable to a surgeon.

Computer-assisted surgical processes according to some embodiments may provide improvements in computing technology through, for example and without limitation, providing visual images and/or information for to facilitate easier, more efficient, more comprehensive, and more accurate evaluations of implantable medical device placement. In contrast, conventional systems are merely able to provide diagnostic images that may not clearly show the implantable medical device within the patient, particularly in combination with device information (for instance, anchor designations, collar positions, and/or the like). Surgical processes according to some embodiments may allow the surgeon to focus on configuring valid implant placement options, while avoiding spending time, effort, and resources on manually tracking or transcribing (i.e., “whiteboarding”) device information. Embodiments are not limited in this context. Additional improvements to computing technology would be known to those of skill in the art based on the present disclosure.

Surgical processes according to some embodiments may be applied to various practical applications. For example, device images and models may be applied by a surgeon and/or computing system to generate a surgical plan. In another example, device images and models may be applied by a surgeon and/or computing system to administer surgery to a patient, such as performing annuloplasty ring implantation as part of a valve repair procedure. In an additional example, device images and models may be integrated into a surgical system hardware and/or software used to plan and/or perform a surgical procedure. Embodiments are not limited in this context. Additional practical applications would be known to those of skill in the art based on the present disclosure.

Further features and advantages of at least some of the embodiments of the present invention, as well as the structure and operation of various embodiments of the present invention, are described in detail below with reference to the accompanying drawings

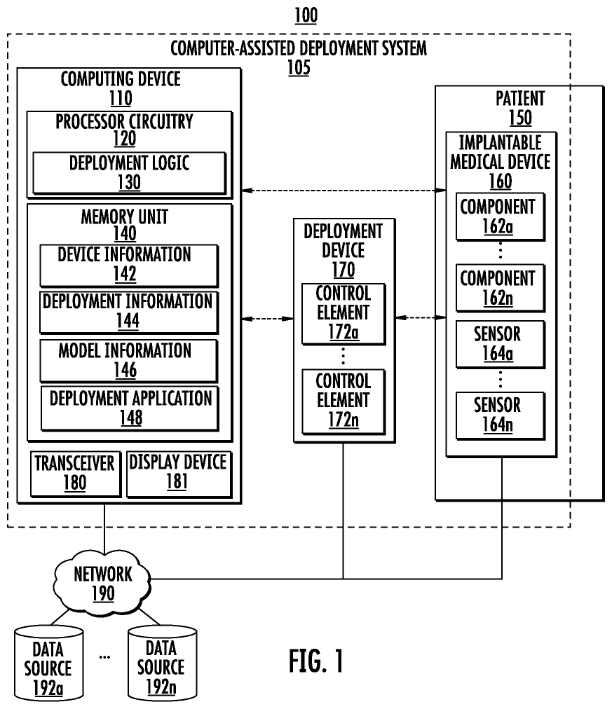

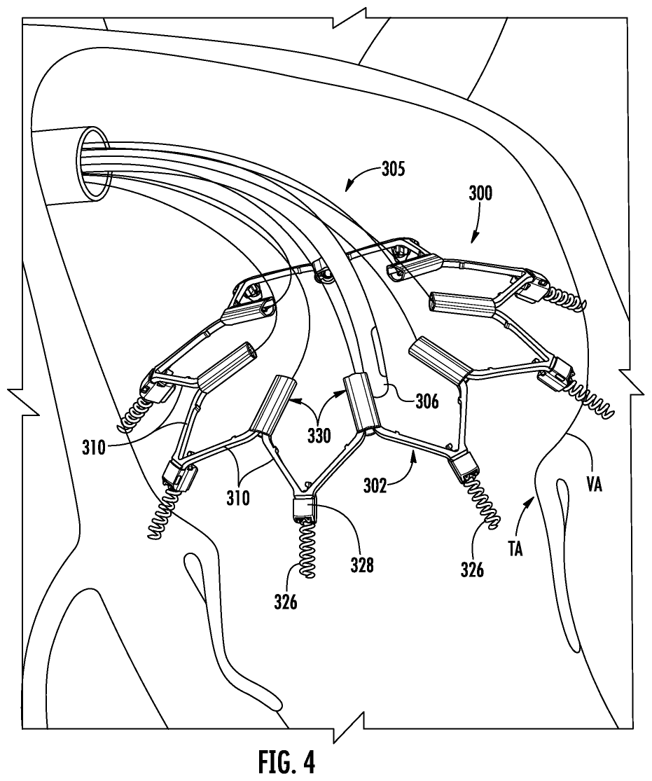

FIG. 1 illustrates an example of an operating environment for a computer-assisted deployment system in accordance with one or more features of the present disclosure. As shown in FIG. 1, operating environment 100 may include a computer-assisted deployment system 105 for delivering, positioning, configuring, implanting or otherwise manipulating an implantable medical device 160 within a patient 150. In some embodiments, implantable medical device 160 may be or may include an annuloplasty ring (see, for example, implantable medical device 300 of FIGS. 3 and 4) for implantation within the heart of a patient. Implantable medical device 160 may include various components 162a-n that may be positioned, configured, implanted, or otherwise manipulated during an implantation procedure. For an annuloplasty ring, components 162a-n may include anchors and/or collars (see, for example, anchors 320 and collars 330 of FIGS. 3 and 4).

One or more sensors 164a-n may be associated with implantable medical device 160 and/or components 162a-n. In some embodiments, sensors 164a-n may determine information associated with implantable medical device 160, such as placement information, configuration information, implantation information, and/or the like. In various embodiments, sensors 164a-n may include image sensors, cameras, diagnostic imaging devices, and/or the like. In one embodiment, sensors may include an imaging catheter, such as an ultrasound catheter or intravascular cardiac echography (ICE) catheter. In exemplary embodiments, sensors 164a-n may include position sensors, force sensors, accelerometer sensors, orientation sensors, and/or any other type of sensor now known or hereafter developed that may measure information associated with implantable medical device 160 and/or components 162a-n. Although sensors 164a-n are depicted as being within implantable medical device 160 in FIG. 1, embodiments are not so limited, as sensors 164a-n may be arranged separate from medical implant device and operate according to the teachings of the present disclosure.

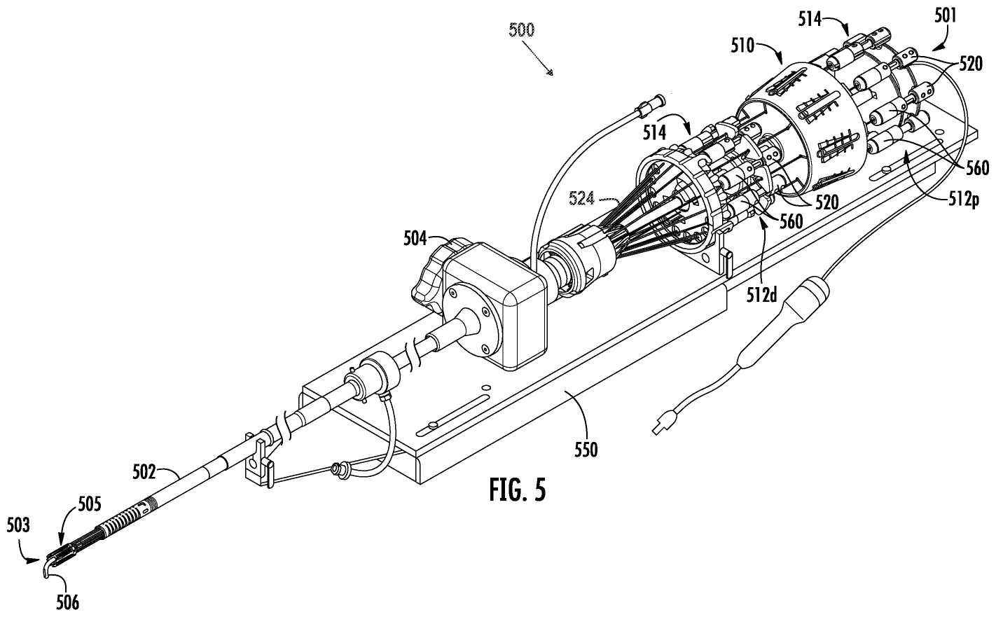

Computer-assisted deployment system 105 may include a deployment device 170 for deploying implantable medical device 160 within patient. For an annuloplasty ring device, deployment device 170 may be the same or similar to delivery/deployment system 500 depicted in FIG. 5. In some embodiments, deployment device 170 may be physically connected to implantable medical device 160 and/or components 162a-n, for example, via wires, shafts, or other structures. In various embodiments, deployment device 170 may be communicatively coupled to medical device 160 and/or components 162a-n through wired or wireless communication protocols. Deployment device 170 may include control elements 172a-n for manipulating implantable medical device 160 and/or components 162a-n. For an annuloplasty ring device, for instance, a control element 172a-n may include a knob, slider, or other structure that may be manipulated to generate a corresponding manipulation of implantable medical device 160 and/or components 162a-n (for instance, moving an anchor position, implanting an anchor in heart tissue, adjusting a collar, and/or the like).

In some embodiments, control elements 172a-n may be adjusted via manual manipulation by an operator (for instance, turning a knob, moving a slider, and/or the like). In some embodiments, control elements 172a-n may be adjusted via computer-based control, for example, automatically via a control system of the deployment device 170 (not shown) and/or operator manipulation (for instance, via pressing a button, entering a value, and/or the like). In exemplary embodiments, implantable medical device components 162a-n may be adjusted via physical adjustment through deployment device 170 (see, for example, FIG. 5). In various embodiments, implantable medical device 160 and/or components 162a-n may be automatically adjusted directly or using deployment device 170 via electronic or servo-based motors, valves, or other adjustment elements. In an automatic-adjustment embodiment, for example, deployment device 170 and/or computing device 110 may send control signals to elements configured to manipulate implantable medical device 160 and/or components 162a-n (for instance, to move and/or rotate implantable medical device 160, thread an anchor, adjust a collar, and/or the like). For example, deployment application 148 may generate control signals to manipulate implantable medical device 160, components 162a-n, deployment device 170, and/or control elements 172a-n.

As will be described herein, the features according to the present disclosure may be used with any suitable implantable device, including annuloplasty rings, now known or hereafter developed. In this regard, the present disclosure should not be limited to the details of the annuloplasty rings, anchors, collars, sensors, deployment device, and/or the like disclosed and illustrated herein unless specifically claimed and that any suitable implantable medical device and/or deployment device can be used in connection with the principles of the present disclosure.

Computer-assisted deployment system 105 may include a computing device 110 that, in some embodiments, may be communicatively coupled to network 190 via a transceiver 180. Computing device 110 may be or may include one or more logic devices, including, without limitation, a server computer, a client computing device, a personal computer (PC), a workstation, a laptop, a notebook computer, a smart phone, a tablet computing device, and/or the like. In some embodiments, computing device 110 may be communicatively coupled to medical device 160, components 162a-n, deployment device 170, and/or control elements 172a-n through wired or wireless communication protocols. Non-limiting examples of communication protocols may include Wi-Fi (i.e., IEEE 802.11), radio frequency (RF), Bluetooth™, Zigbee™, near field communication (NFC), Medical Implantable Communications Service (MICS), universal serial bus (USB), Lightning, serial, and/or the like.

Computing device 110 may include a processor circuitry 120 that may include and/or may access various logics for performing processes according to some embodiments. For instance, processor circuitry 120 may include and/or may access a deployment logic 130. Processing circuitry 120 and/or deployment logic 130, and/or portions thereof may be implemented in hardware, software, or a combination thereof. As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution. For example, a logic, circuitry, or a module may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a system-on-a-chip (SoC), memory units, logic gates, registers, semiconductor device, chips, microchips, chip sets, software components, programs, applications, firmware, software modules, computer code, a control loop, a computational model or application, an AI model or application, an ML model or application, a proportional-integral-derivative (PID) controller, FG circuitry, variations thereof, combinations of any of the foregoing, and/or the like.

Although deployment logic 130 is depicted in FIG. 1 as being within processor circuitry 120, embodiments are not so limited. For example, deployment logic 130 and/or any component thereof may be located within an accelerator, a processor core, an interface, an individual processor die, implemented entirely as a software application (for instance, deployment application 148) and/or the like.

Memory unit 130 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In addition, memory unit 130 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid state drive (SSD), and/or the like.

Memory unit 130 may store various types of information and/or applications for an adjustment compliance process according to some embodiments. For example, memory unit 130 may store device information 142, deployment information 144, model information 146, and/or a deployment application 148. In some embodiments, device information 142, deployment information 144, model information 146, deployment application 148, and/or portions thereof, may be stored in one or more data stores 192a-n accessible to computing device 110 via network 190.

In some embodiments, device information 142 may include information associated with implantable medical device 160, components 162a-n, sensors 164a-n, deployment device 170, and/or control elements 172a-n. For example, device information 142 may include a type of implantable medical device, such as an annuloplasty ring, and any associated information, such as dimensions, anchor information, collar information, sensor information, and/or the like. In general, device information 142 may include any information, data, files, images, and/or the like associated with implantable medical device 160 and/or deployment device 170 that may be used for surgical processes according to some embodiments.

In various embodiments, deployment information 144 may include information associated with a deployment of implantable medical device 160 and/or components 162a-n. For example, for an annuloplasty ring, deployment information 144 may include position information associated with a position of implantable medical device 160 and/or components thereof (e.g., anchor position (relative to other anchors and/or anatomical features), implantable medical device 160 position, and/or the like) and/or configuration information or settings (e.g., configuration of implantable medical device 160 and/or components thereof, including, without limitation, anchor implantation information (e.g., whether an anchor is implanted within tissue, depth of anchor implantation, and/or the like), collar information (e.g., adjustment values)), and/or the like. In general, position information relates to a position of implantable medical device 160 and/or components thereof and configuration information relates to non-positional configurations or settings of implantable medical device 160 and/or components thereof. For example, a camera or other sensor 164a-n may be used to determine the position of an anchor, including, for instance, a position about the heart valve or a position relative to a component of implantable medical device 160. In another example, deployment information 144 may include video images, still images, diagnostic images, diagnostic video, and/or the like captured via sensors 164a-n. In exemplary embodiments, deployment information 144 may include feedback information associated with performance of patient anatomy and/or implantable medical device 160. For example, for a valve repair procedure, the feedback information may include regurgitation information and/or patient vitals. In general, deployment information 144 may include any information associated with a current deployment status of implantable medical device 160 and/or components 162a-n.

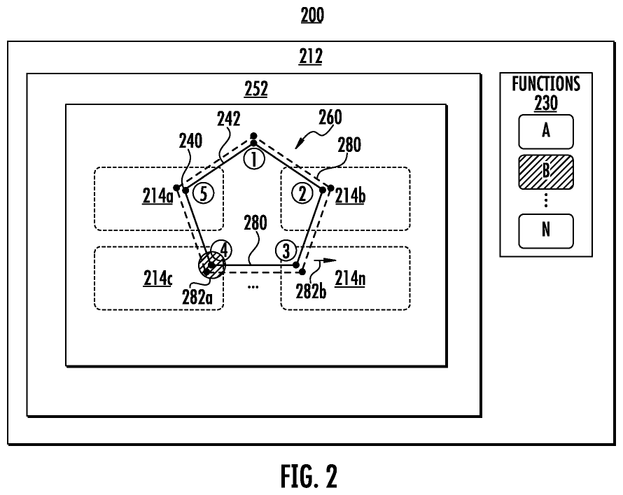

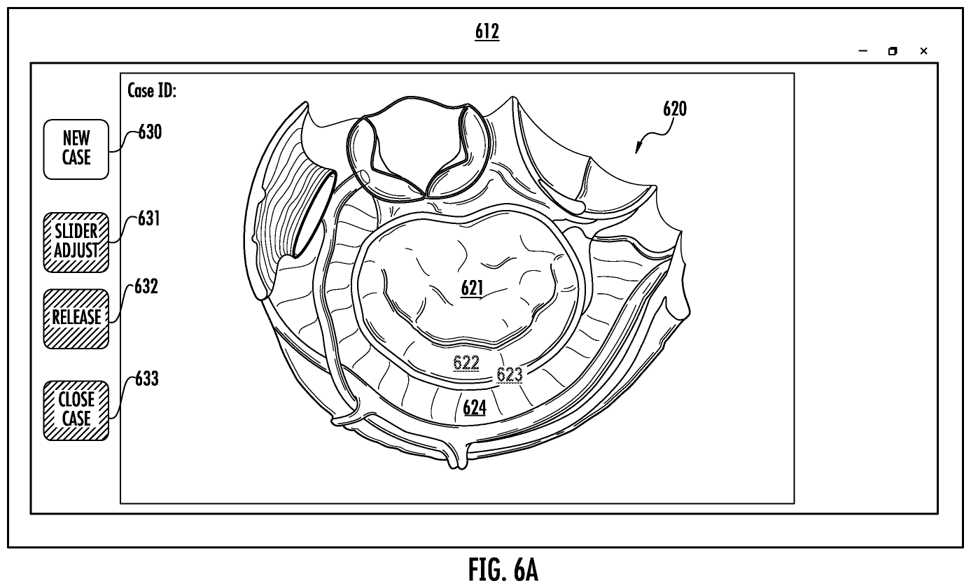

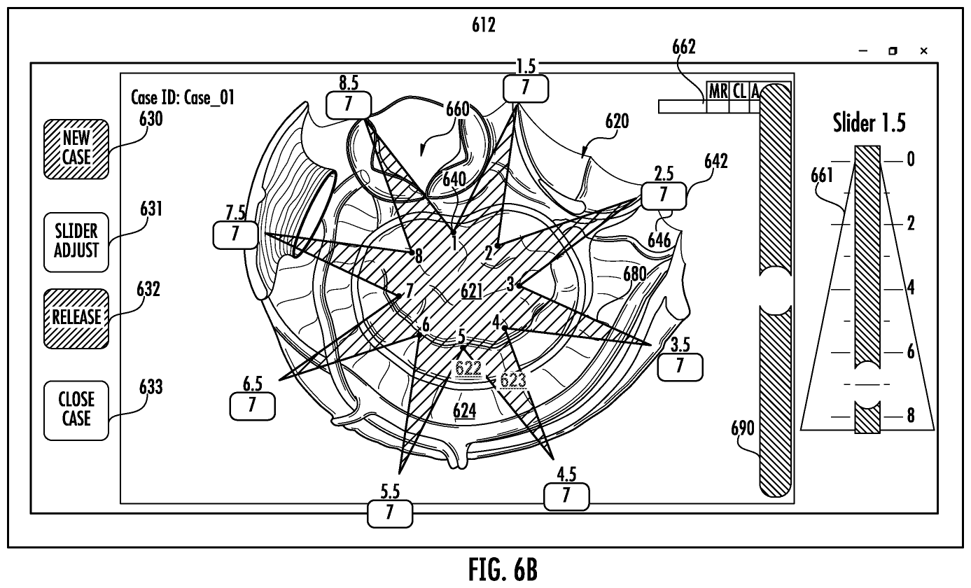

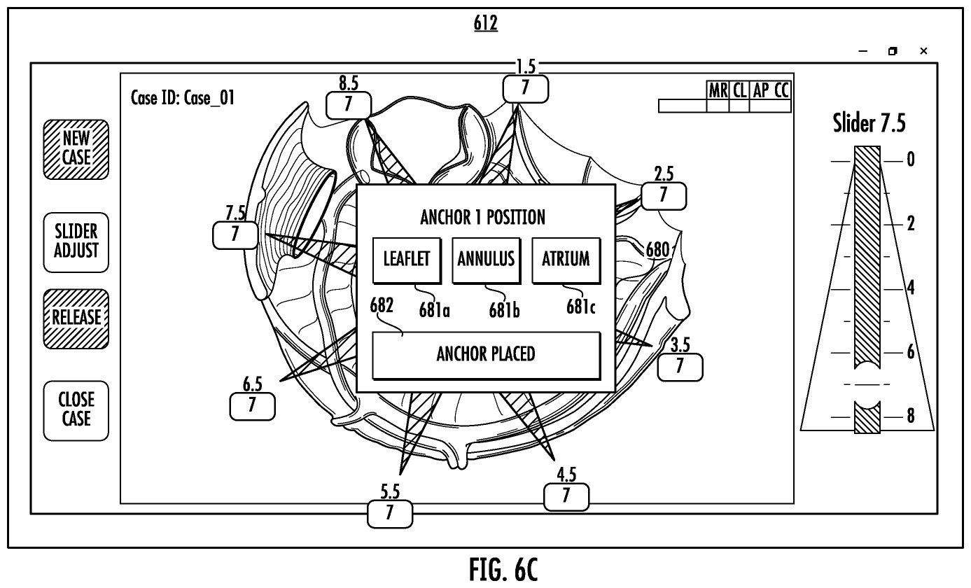

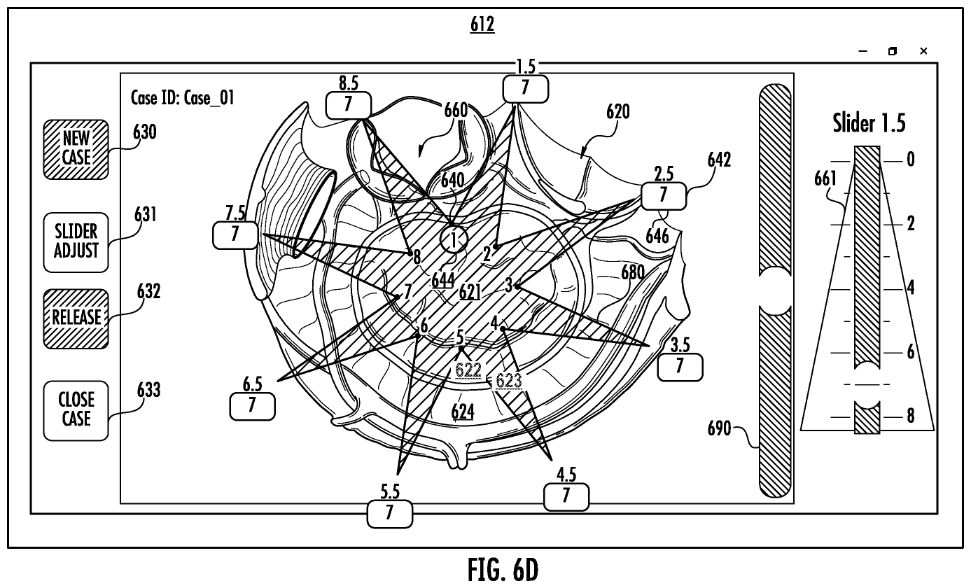

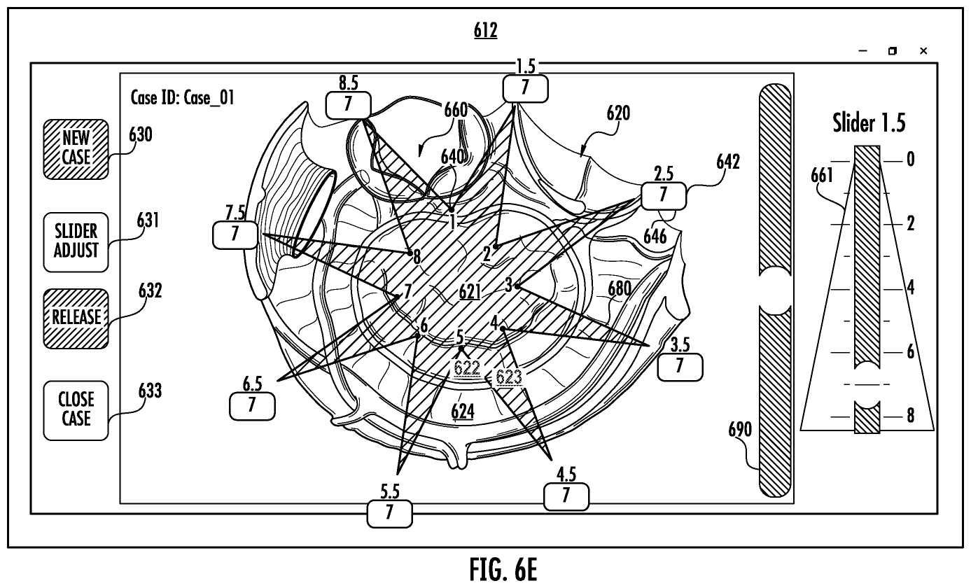

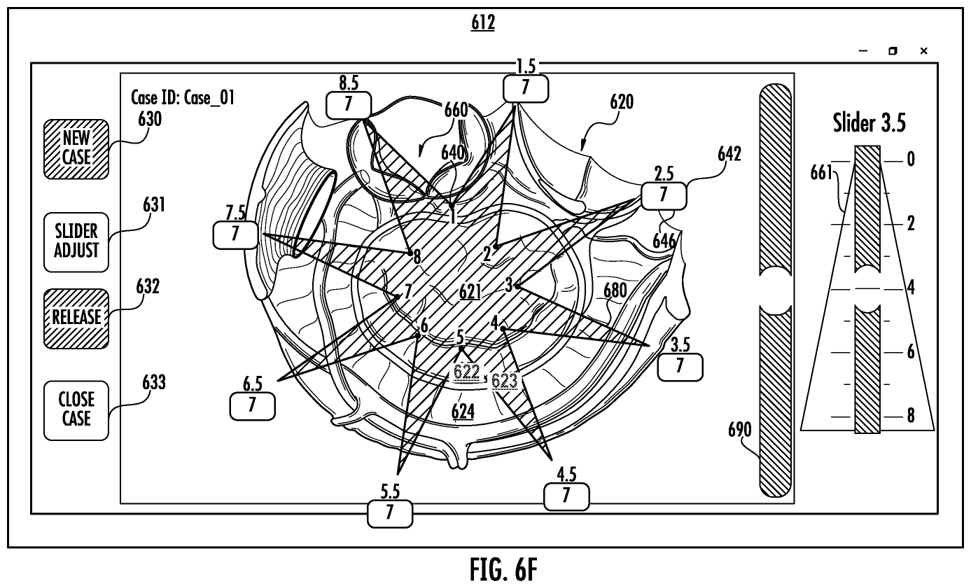

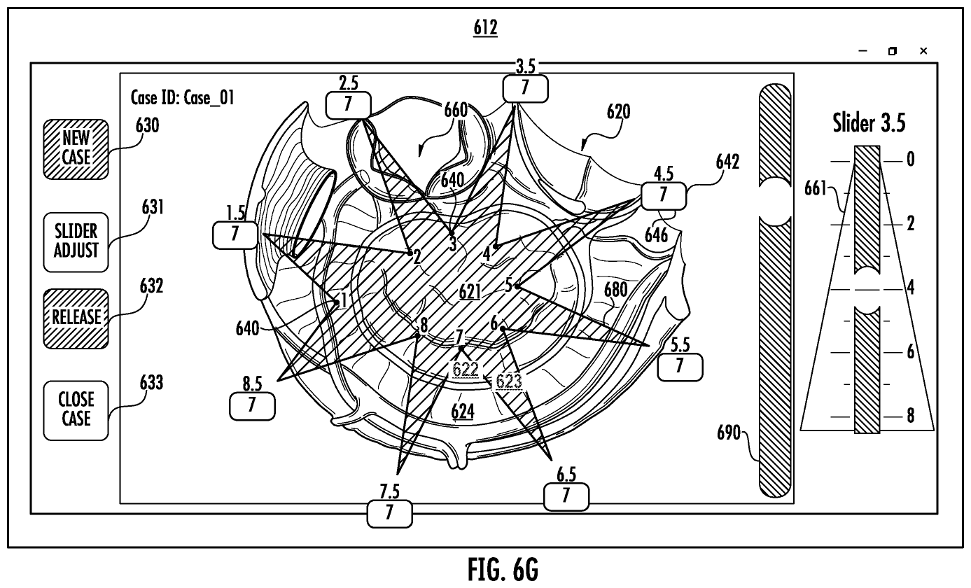

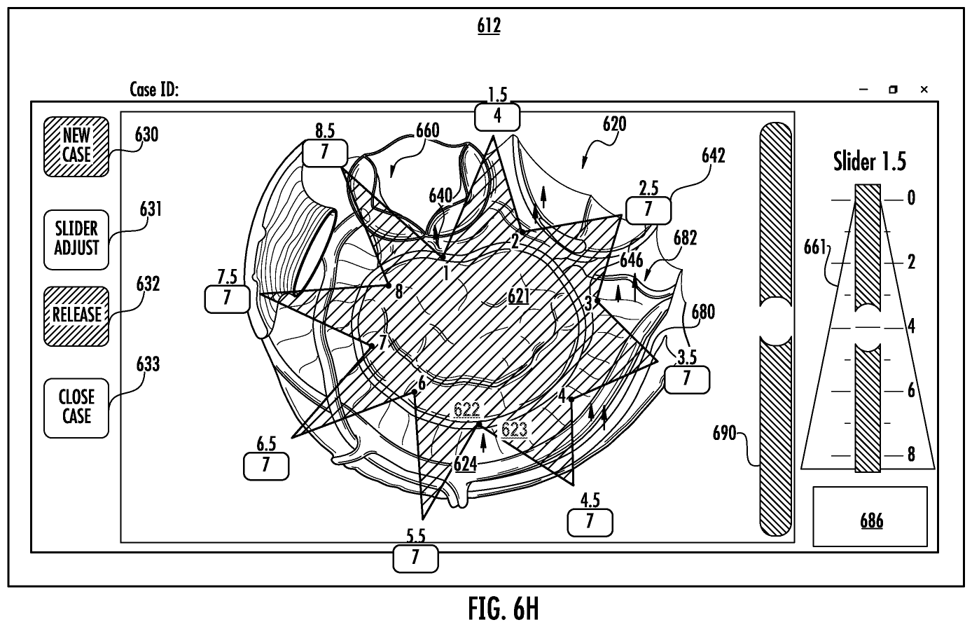

In exemplary embodiments, model information 146 may include information associated with computational models, historical information, and/or the like that may be used to deliver, position, implant, adjust, or otherwise manipulate implantable medical device 160 and/or components 162a-n. In some embodiments, model information 146 may include machine learning (ML), neural network (NN), and/or other artificial intelligence (AI) models that may be trained to determine optimized placement information for implantable medical device 160 and/or components 162a-n. In various embodiments, the computational models may be trained based on information associated with a population of patients with the same or similar surgical procedures, implants, anatomy, and/or the like. For example, a NN or other ML model may be trained to receive input in the form of a device information 142 and patient information (including information determined during a surgical procedure) and determine one or more optimized installation configurations, for instance, based on outcomes of a population of patients. In one example, an implanted implantable medical device may be associated with a shape formed by a portion of the device (see, for example, FIGS. 2 and 6B).

In another example, components of an implanted implantable medical device may be associated with coordinates, for example, relative to other components and/or patient anatomy (for instance, one or more anchors of an annuloplasty ring may have a coordinate position relative to a standard position, such as a central point of the heart valve, annuloplasty ring device, or other object). Coordinates, shapes, or other implant configuration information may be determined and stored for a population of patients (for instance, as historical implant configuration information) along with patient information (for instance, physiological information of the patient and/or patient anatomy, such as heart valve characteristics, dimensions, and/or the like) and outcome information (for instance, patient mortality, patient morbidity, regurgitation information, and/or the like) may be stored as model information. One or more computational models may be trained to determine an optimized configuration (for instance, a shape-based, coordinate-based, and/or based on another configuration structure) for implantable medical device 160 based on the patient information.

In some embodiments, a surgical process may be performed, for instance, with automatic-adjustment steps, surgeon-adjustment steps, or a combination thereof, to install implantable medical device 160 to conform to the optimized configuration. In another example, an optimized configuration image may be presented to the surgeon via deployment application 148 such that the surgeon may attempt to install implantable medical device 160 to match or otherwise correspond to features of the optimized installation image.

Deployment logic 130, for example, implemented via deployment application 148 being executed by processor circuitry 120, may operate to perform a surgical process to facilitate proper implantation and configuration of implantable medical device 160 within patient 150. In some embodiments, deployment application 148 may be or may include an application being executed on computing device 110 (including a mobile application or “app” executing on a mobile device form factor).

FIG. 2 illustrates an example of an operating environment for a computer-assisted deployment application that may be representative of some embodiments. As shown in FIG. 2, operating environment 200 may include user interface 212 that may be generated via deployment logic 130 (for instance, as a result of executing deployment application 148). User interface 212 may be presented, for example, via display device 181 of computing device 110.

User interface may include a surgical window or form 252 configured to provide a live or real-time view, simulated view, or a combined live/simulated view of a surgical process for positioning, configuring, implanting, or otherwise manipulating an implantable medical device 260. An anatomical region 220 and segments 214a-c of anatomical region 220 may be presented within surgical window 252. For example, for a heart valve repair procedure, a portion of the heart (for instance, a heart valve and adjacent tissue, such as leaflets, the annulus, and/or the atrium) may be presented as the anatomical region. In various embodiments, segments 214a-c of anatomical region may be determined and classified for individual depicting within surgical window 252. For example, the leaflets, the annulus, and/or the atrium may be detected, for instance, via image recognition and/or user input, and individually designated within surgical window.

In some embodiments, anatomical region 220 may be an actual image or video of patient anatomy based on a camera or medical diagnostic image. In various embodiments, anatomical region 220 may be a simulated image determined based on user input and/or captured information, such as camera or medical diagnostic images. In exemplary embodiments, anatomical region may be a combination of real-time images and simulated images, for example, in an overlay configuration. Embodiments are not limited in this context.

In various embodiments, implantable medical device 260 may be depicted within surgical window 252. Implantable medical device 260 may be an actual image or video of patient anatomy based on a camera or medical diagnostic image. In various embodiments, anatomical region 220 may be a simulated image determined based on user input and/or captured information, such as camera or medical diagnostic images. For example, implantable medical device 260 may be a simulated image or mapping generated based on configuration information determined for a physical implantable medical device positioned within a patient. In another example, implantable medical device 260 may be a real-time image or real-time composite image based on one or more camera or diagnostic images. In some embodiments, implantable medical device 260 may be an image based on a standard image pre-generated for the particular type of device. In other embodiments, a surgical process may include a registration process in which sensor information is collected for the physical device (and/or patient anatomy) to generate a corresponding image model to be used for implantable medical device 260 (and/or anatomical region). Embodiments are not limited in this context.

In some embodiments, surgical window 252 may provide a more comprehensive view of implantable medical device 260 than provided via sensor images. For example, a conventional view of an annuloplasty ring may be via diagnostic imaging from within the center of the ring frame. It may be difficult for a surgeon to easily and accurately determine the position and installation status of the annuloplasty ring and components thereof, such as anchors and collars. In some embodiments, the view of implantable medical device 260 may be a top-down view mapped or otherwise generated via extrapolating sensor information (see, for example, FIG. 6B) to generate a simulated image. In this manner, a surgeon may be able to more efficiently and accurately perform an implantation procedure. In some embodiments, the image or mapping of implantable medical device 260 may be overlaid on top of a real-time or simulated image of patient anatomy 220.

In various embodiments, implantable medical device 260 may have a shape or pattern 270. In one example, for an annuloplasty ring, the a top-down view of the annuloplasty ring may have a generally star shape (see, for example, FIGS. 3 and 6B). Accordingly, the frame of an annuloplasty ring (see, for example, frame member 302 of FIG. 3) formed by the anchors and connecting struts may form a shape. In general, shape 270 may result from the position of vertices 240 (for instance, anchors (see, for example, anchors 320 or apices 315 of FIG. 3)) connected by edges 242 (see, for example, struts 310 of FIG. 3). Although multiple vertices 240 and edges 242 are depicted in FIG. 2, only one of each is designated to simplify the figure.

In some embodiments, components of implantable medical device 260 may have coordinates based on a standard reference, such as a central point of the implantable medical device 260, a reference part of the anatomy, and/or the like. For example, each vertices 240 may have a coordinate (for instance, a distance from a reference point, an angle from a reference point, combinations thereof, and/or the like).

In some embodiments, shape 270 and/or coordinates may be used to determine an optimized configuration. For example, model information 246 may be used to determine an optimized configuration of implantable medical device 260 within patient anatomy 220. In various embodiments, an optimized shape (or shape range) and/or optimized coordinates (or coordinates range) may be determined for a particular patient anatomy. The surgical procedure may be performed to place implantable medical device 260 in a shape and/or coordinates that matches or otherwise conforms with the optimized shape, including, for example, placement of the components of implantable medical device on or in optimized segments 214a-c and/or configuration settings for components (for instance, collar adjustment values).

In various embodiments, indicators 282 may be displayed within surgical window 252 to provide indications for placement and/or configuration of components of implantable medical device 260. For example, portions of implantable medical device 260 (for instance, a vertex, an anchor, a collar, and/or the like) may be highlighted 282a to indicate that movement or adjustment of the portion should be performed. For example, indicator 282a may indicate that an anchor of an annuloplasty ring is in a position where it will not properly hit tissue (or at a sufficient depth) when rotated down to be installed within the valve. In another example, indicator 282a may highlight a collar of an annuloplasty ring that should be adjusted to increase/decrease the cinching of the annuloplasty ring. In a further example, an arrow 282b may be used to indicate a direction and/or an amount of movement suggested for a component of implantable medical device 260 based on deployment information 244 and/or an optimized configuration. For example, arrow 282b may be displayed to indicate that vertex 240-3 (i.e., anchor 3) should be moved to the left by a certain amount (for instance, 5 mm).

In various embodiments, an optimized configuration element 280 may be overlaid or otherwise presented in combination with patient anatomy 220. In some embodiments, optimized configuration 280 may present an optimized configuration of components of implantable medical device to achieve an optimized implantation. In some embodiments, a surgeon may manipulate implantable medical device 260 to match or otherwise conform with optimized configuration 280. In some embodiments, optimized configuration 280 may include configuration information, such as collar adjustment information.

In some embodiments, user interface 212 may include functions 230, for example, for manipulating user interface 212 and/or implantable medical device 260. For example, in various embodiments, the position and/or configuration of medical implant device 260 and/or components thereof may be based, at least in part, on user input. A function 230 may be used to input configuration information (for instance, a region 214a-n (e.g., annulus) for placement of a vertex (e.g., anchor), adjustment of a collar, and/or the like). In some embodiments, a function 230 may operate to control and manipulate deployment device 170 and/or implantable medical device 160. For example, selection of a function 230 to move an anchor of an annuloplasty ring to a certain region 214a-n (for instance, annulus), may cause a corresponding control element 172a-n to manipulate the position of anchor to the specified region 214a-n. In another example, selection of a collar adjustment value via a function 230 may cause a corresponding control element 172a-n to manipulate the collar up or down to meet the adjustment value.

FIGS. 3 and 4 illustrate a perspective view of an example of an implantable device configured for delivery at an implantation site by a delivery/deployment system. Implantable device 300 in this example is an implantable device for annuloplasty, such as for custom reshaping of a heart valve (e.g., the mitral valve, as illustrated, or the tricuspid valve), and is capable of moving between the collapsed and expanded configurations and positions therebetween to modify the shape of the valve annulus VA at which it is implanted/to which it is secured. An imaging catheter 306 may be used to locate the treatment site TS at which implantable device 300 is to be delivered/deployed and implanted and/or to observe the configuration and/or position of the implantable device 300 during implantation and adjustment. Once at the treatment site TS, implantable device 300, which may be held in a compressed or retracted or unexpanded (such terms being used interchangeably herein without intent to limit) configuration by a retention device or by the delivery device or otherwise, is allowed to expand for deployment and placement and implantation, as illustrated in FIG. 4. Expansion may occur naturally, for example if the frame is formed of a shape memory or super elastic material (e.g., Nitinol) that is biased towards an expanded state. In alternate embodiments, expansion may be mechanically controlled, for example through the use of a force applied within the frame using an expandable deployment device (e.g., an inflatable balloon or the like).

With reference to FIGS. 3 and 4, the illustrated example of implantable device 300 includes a frame member 302 that may be disposed about a heart valve or other cardiac feature. Frame member 302 may be generally symmetrical with respect to the central frame axis FA although it need not be symmetrical. Frame member 302 may form a generally tubular shape, the term “tubular” being understood herein to include circular as well as other rounded or otherwise closed shapes. Frame member 302 may be configured to change shape, size, dimension, and/or configuration. For example, frame member 302 may assume various shapes, sizes, dimensions, configurations etc. during different phases of deployment such as during pre-delivery, delivery, tissue engagement, anchoring, cinching, etc.

Frame member 302 may be formed from one or more struts 310 that may form all or part of frame member 302. Struts 310 may include elongated structural members formed of a metal alloy, a shape memory material, such as an alloy of nickel titanium or other metals, metal alloys, plastics, polymers, composites, other suitable materials, or combinations thereof. In one embodiment, struts 310 may be formed from the same, monolithic piece of material (e.g., tube stock). Thus, reference to struts 310 may refer to different portions of the same, extensive component. Alternatively, reference to struts 310 may refer to components that are formed separately and attached together (optionally permanently, such as by welding or other methods). In some embodiments, struts 310 may be separate components that are detachably coupled to form proximal apices 314 and distal apices 315. Alternatively, if formed from a monolithic piece of material, the material may be cut or otherwise formed to define proximal apices 314 and distal apices 315. Frame member 302 may be considered to be substantially tubular, and configured to change shape, size, dimensions, and/or configuration. For example, frame member 302 may assume various shapes, sizes, dimensions, configurations etc. during different phases of deployment such as during pre-delivery, delivery, tissue engagement, anchoring, and adjustment (e.g., cinching).

As shown in FIGS. 3 and 4, in the illustrated example of an embodiment of an implantable device 300, a plurality of anchors 320 are carried at a distal end 303 of the frame member 302, such as along distal apices 315 of frame member 302, and a plurality of collars or cinch collars or sleeves or cinch sleeves sliders or nuts 330 (such terms being used interchangeably herein without intent to limit, reference being made generally to collars for the sake of convenience) are carried at proximal apices 314 of frame member 302. In the illustrated embodiments, proximal end 301 of frame member 302 is directed proximally toward and engaged or carried by the delivery/deployment system 500 (see FIG. 5), and distal end 303 of frame member 302 extends distally from delivery/deployment system 500 and is the end engaged with the treatment site TS. It will be appreciated that alternate configurations of frame member 302, such as depending on the manner and orientation in which the implantable device 300 is delivered, are within the scope and spirit of the present disclosure.

An example of a system 500 for delivery and/or deployment of (or other action associated with) an implantable device 300 (carried by or coupled to or otherwise associated with delivery/deployment system 500 and which may be considered in some instances as part of delivery/deployment system 500) to a treatment site is shown, in a perspective view, in FIG. 5. System 500 will be referenced herein as a delivery/deployment system 500 to convey the optional multi-use aspect of the system without intent to limit the system to a single or particular use in connection with an implantable device 300. Moreover, references to delivery/deployment systems are to be understood as optionally including an implantable device. The delivery/deployment system 500 may include a steerable delivery device 502 (e.g., catheter, sheath, or the like) through which implantable device 300 may be delivered (e.g., transluminally), and which may be controlled (e.g., steered or navigated) by a delivery device control knob 504.

System 500 may include one or more devices for imaging capabilities, such as an imaging catheter 506, such as an ultrasound catheter or intravascular cardiac echography (ICE) catheter. An example of a steerable delivery device and system with various positioning and imaging capabilities is described in U.S. Pat. No. 10,335,275, titled “Methods for Deployment of Heart Valve Devices Using Intravascular Ultrasound Imaging”, and issued on Jul. 2, 2019, which patent is incorporated herein by reference in its entirety for all purposes. A control handle assembly 510 is provided at a proximal end 501 of delivery/deployment system 500. A stage or stand 550 may be provided to support control handle assembly 510. Control handle assembly 510 includes one or more control sections 512, and may also include delivery device control knob 504. Each control section 512 may include one or more knobs 514. Each knob may be configured for and capable of controlling (e.g., steering or operating) a different component of delivery/deployment system 500. Different knobs 514 may be provided to effect or implement different operations or actions or control movements on the implantable device 300. Although control sections 512 are illustrated as a proximal control sections 512p and a distal control section 512d, other relative arrangements of control sections 512 (such as peripherally with respect to one another) are within the scope of the present disclosure.

In some embodiments, each control section 512 has at least one control knob 514 configured and arranged for controlling at least one component of delivery/deployment system 500 and/or at least one component at a distal end 503 of the delivery/deployment system 500. Latch 505, on a distal end 503 of flexible elongate member 524 (with the latch knob 520 on the proximal end of the flexible elongate member 524), is configured to be engaged, coupled, or otherwise with a corresponding element (latch or coupler or connector or the like) on implantable device 300 (examples being described in further detail below). Latch knob 520 may be manipulated, moved (rotatably, axially, etc.), or otherwise to manipulate, move, etc. (such terms being used interchangeably herein without intent to limit) latch 505 in or out of engagement with the corresponding element on implantable device 300, or to manipulate, move, etc. implantable device 300, as will be described below in further detail with reference to an example of an implantable device 300.

In some embodiments, some of knobs 514 are coupled with other components of delivery/deployment system 500. For instance, a knob may be provided to actuate or control an actuator or driver or driver shaft (such terms being used interchangeably herein without intent to limit) coupled to implantable device 300 to manipulate or operate or actuate or control (such terms being used interchangeably herein without intent to limit) a component of implantable device 300, such as to implant implantable device 300. In some embodiments, as described in further detail below, one or more knobs may be provided to control one or more driver shafts configured to transmit torque to a component of an implantable device 300 (e.g., an anchor 320 or a collar actuator 336 of a collar 330) to implant and/or adjust or otherwise manipulate or move implantable device 300.

In accordance with various principles of the present disclosure, various embodiments of flexible elongate member 524 include at least one locking region (not shown) formed along an exterior thereof. In some embodiments, such locking region has a shape and/or configuration distinct from the surrounding exterior contour of the flexible elongate member 524 to facilitate engagement or coupling of flexible elongate member 524 with another component, such as a latch knob 520 or a latch 505 (e.g., for coupling or mounting of latch knob 520 or the latch 505 with or on the flexible elongate member 524). The locking region may be formed in various manners which modify the exterior of flexible elongate member 524, such as, without limitation, stamping, swaging, grinding, machining, or otherwise shaping features into wire, or using profile drawn wires and shafts tubing.

In some embodiments, one or more components of system 500 may be automatically operated via control signals, for example, received at system 500 from computing device 110. For example, execution of deployment application 148 according to some embodiments may cause control signals to manipulate system 500 to cause a corresponding movement or configuration adjustment of implantable medical device 300.

As may be seen with reference to FIGS. 4 and 5, one or more latches of the delivery/deployment system 500 may engage a corresponding anchor latch 322 on a respective anchor head 324 on frame member 302, and one or more latches 505 may engage a corresponding collar actuator latch 332 on a collar actuator head 334 of a collar actuator 336. The latches 322 and 332 on frame member 302 are configured to engage with the latches of delivery/deployment system 500 to couple the associated components together such as for delivery of implantable device 300 to the treatment site TS. As may further be seen with reference to FIGS. 3-5, one or more drivers 305 of the delivery/deployment system 500 may engage a respective anchor head 322 on frame member 302, and one or more drivers 305 may engage a collar actuator head 334 of collar actuator 336. Drivers 305 may be coupled to system 500 via actuator knobs 560. The drivers 305 typically extend over latches 322, 332, such as to maintain engagement of latches 322, 332 with implantable device 300 (such as during delivery and/or manipulation of frame member 302 by the delivery/deployment system 500). Actuator knob(s) 160 may be manipulated, moved (rotatably, axially, etc.), or otherwise to manipulate, move, etc. (such terms being used interchangeably herein without intent to limit) driver 305 to effect the desired action on the implantable device 300. Rotation of drivers 305 coupled to the anchor heads 324 causes advancement or withdrawal of anchor shafts 326 of anchors 320 with respect to treatment site TS (in this example, a valve annulus) to implant, remove, or adjust the position of frame member 302. In some embodiments anchors 320 may translate through an anchor housing 328 coupled to frame member 302. The anchor shaft 326 (such as in the form of a helical shaft) may be coupled to and extend through a portion of an associated distal apex 315, with or without an associated anchor housing 328. Rotation of a driver coupled to a collar actuator head 334 causes advancement or withdrawal of collar 330 with respect to proximal apex 314 over which collar 330 is positioned to adjust the relative positions of the struts 310 joined at such apex. Such adjustment results in adjustment of at least one of the size, shape, configuration, dimension, etc. of frame member 302 (e.g., retraction/compression or expansion of the frame upon bringing adjacent struts 310 closer or further apart, respectively) to affect at least one of the size, shape, configuration, dimension, etc. of the treatment site TS (such as to restore or correct the shape of a valve annulus for proper functioning or competency thereof).

Although embodiments of the present disclosure may be described with specific reference to an implant for use with mitral valves or tricuspid valves, it is appreciated that various other implants may similarly benefit from the systems and methods disclosed herein. For example, implants which must withstand the palpatory forces for repairing a tricuspid valve annulus and/or addressing other dilatation, valve incompetency, valve leakage and other similar heart failure conditions may also benefit from the concepts disclosed herein. Principles of the present disclosure may be applied to other delivery/deployment devices subject to torsional forces. Principles of the present disclosure may be applied to other delivery/deployment systems and/or implants, such as those disclosed in U.S. Pat. No. 10,575,853, issued Mar. 3, 2020, and titled “Embolic Coil Delivery and Retrieval”; U.S. Pat. No. 10,548,605, issued Feb. 4, 2020, and titled “Detachable Implantable Devices”; and U.S. Pat. No. 10,478,192, issued Nov. 19, 2019, and titled “Detachable Mechanism for Implantable Devices”; each of which patents is incorporated by reference herein in its entirety for all purposes.

FIGS. 6A-6H illustrate an example computer-assisted deployment system configurations for an annuloplasty ring implantation procedure that may be representative of some embodiments. As shown in FIG. 6A, a user interface 612 may depict a heart 620 (or portion thereof) having various regions, such as a valve 621, leaflet 622, annulus 623, and/or atrium 624 as well as other anatomical features. In some embodiments, heart 620 may be a simulated image. In various embodiments, heart 620 may be a simulated image based on information obtained from the patient, such as sensor information, diagnostic images, and/or the like. In exemplary embodiments, heart 620 may be an actual image of a portion of a heart of the patient obtained using a camera or diagnostic imaging device (for instance, an ICE system). In some embodiments, heart (or heart portion, for example, a region of a heart valve) 620 may be an image formed from combining or overlaying actual and simulated images. User interface 612 may include various functions, such as a New Case function 630, Slider Adjust function 631, Release function 632, and/or Close Case function 633.

Referring to FIG. 6B, selection to open a new case may cause an annuloplasty ring 660 to be presented on user interface 612, for example, overlaid over heart 620. In some embodiments, annuloplasty ring 660 may be a simulated image intended to represent an actual annuloplasty ring arranged within the heart of the patient. In various embodiments, annuloplasty ring 660 may be a real-time image captured via a camera or diagnostic imaging device (for instance, an ICE device). In exemplary embodiments, heart 620 may be a simulated image and annuloplasty ring 660 may be a simulated image, heart 620 may be a real-time image and annuloplasty ring 660 may be a simulated image, heart 620 may be a simulated image and annuloplasty ring 660 may be a real-time image, or heart 620 may be a real-time image and annuloplasty ring 660 may be a real-time image.

Annuloplasty ring 660 may be depicted with anchors 640 1-8 and collars 642 1.5-8.5. Although multiple anchors and collars are depicted in FIGS. 6B and 6D-6H, only one of each are labeled to simplify the figure. Reference to a particular anchor will be written as 640-x, with “x” referring to a particular anchor number; reference to a particular collar may be written as 642-y, with “y” referring to a particular collar number. Collars are referenced with a “half” or 0.5 designation to indicate which anchors the collar is arranged between. For example, collar 1.5 is arranged between anchor 1 and anchor 2, collar 2.5 is arranged between anchor 2 and anchor 3, and so on.

In some embodiments, simulated images may be determined, at least in part, based on user input. For example, a standard heart 620 and/or hear annuloplasty ring 660 image may be presented on user interface 612 and an operator may provide input to set up annuloplasty ring 660 to visually emulate the actual annuloplasty ring 660 arranged within the heart of the patient. For example, a user may select an anchor 640 to select a region for placement on heart 620. In reference to FIG. 6C, selection of anchor 640-1 may cause presentation of anchor position window 680. Selection of an anchor position 681a-c may set selection of anchor 640-1 to that position. For example, in FIG. 6B, anchor 640-1 is arranged on the leaflet. Selection of Annulus 681b on anchor position window 680 may place anchor 640-1 on the annulus as depicted in FIG. 6D. In another example, selection of Atrium 681c on anchor position window 680 may place anchor 640-1 on the atrium as depicted in FIG. 6E. In a further example, selection of Anchor Placed 682 may indicate that anchor 640-1 has been placed or affixed to heart 620, as indicated by a circle 644 arranged around anchor 640-1, as shown in FIG. 6D.

Each collar 642 may be associated with an adjustment value 646 (for example, in FIG. 6B, collar 642-2.5 has an adjustment value of 7) corresponding to a slider adjustment setting from a corresponding deployment device (see, for example, FIG. 5). Selection of Slider Adjust 631 for collar 642-1.5 may cause presentation of a slider adjust object 661 to allow an operator to change the adjustment value for a particular collar 642. For example, in reference to FIG. 6H, adjustment value 646 for slider 642-1.5 has been changed via slider adjust object 661 from a value of 7 to a value of 4.

Referring to FIG. 6E, annuloplasty ring 660 may be rotated within user interface 612. For example, an operator may select a portion of annuloplasty ring 660 and rotate annuloplasty ring 660 clockwise or counterclockwise to change a position of anchors 640 about heart 620. In another example, a rotation object 690 may be used to rotate annuloplasty ring 660 (for instance, movement of a slider object on rotation object 690 “up” may cause rotation of annuloplasty ring 660 a corresponding number of degrees in a first direction, and movement of the slider object on rotation object 690 “down” may cause rotation of annuloplasty ring 660 a corresponding number of degrees in a second direction, opposite the first direction).

Referring to FIG. 6H, some embodiments may provide various indicators on user interface 612 to assist in guiding a surgeon with placement and/or configuration of annuloplasty ring 660. For example, an arrow indicator 672 may be used to indicate that movement of a component of annuloplasty ring 660 is suggested to achieve an optimized configuration. For example, in FIG. 6H, arrow indicator 672 indicates that anchor 640-3 should be moved. In some embodiments, information, such as a size, color, or other characteristic of arrow indicator 672 may indicate an amount of movement (for instance, a small movement, a large movement, a particular distance (for instance, 4 mm), and/or the like). In another example, a highlight indicator 674 may be used to indicate a suggested or required adjustment of a component of annuloplasty ring 660. In some embodiments, highlight indicator 674 may indicate that a position, configuration, or other implantation characteristic of a component violates a rule, guideline, goal, and/or the like.

In FIG. 6H, for example, anchors 640-1 and 640-5 are located on the mitral annulus, anchors 640-2, 640-3, and 640-4 are outside the mitral annulus (atrial), and anchors 640-6, 640-7, and 640-8 are located inside the mitral annulus (leaflet). In addition, anchors 640-6, 640-7, and 640-8 are located on the mitral leaflet, anchors 640-1 and 640-7 are located just above the annulus, and anchors 640-2, 640-3, and 640-4 are located so far above the annulus that reach anchors will likely not hit tissue when actuated. Accordingly, an operator may determine based on the configuration of annuloplasty ring 660 that an adjustment is needed, such as moving or swinging the device laterally to get the annuloplasty ring 660 in the proper position. After the adjustment, the sensor data may be refreshed, such as via another ICE sweep. In this manner, implantation of the annuloplasty ring 660 via a surgical process according to some embodiments may be performed via a methodical approach that is more efficient and less error prone than conventional implantation techniques.

In some embodiments, performance information 686 may be provided to indicate performance metrics of heart 620 and/or portions thereof. For example, performance information 686 may include feedback, such as a regurgitation rate, heart rate, blood pressure, and/or other physiological information of patient based on the placement and/or configuration of annuloplasty ring 660 and/or components thereof. In some embodiments, performance information 686 may include actual measurements from the patient, simulated values based on historical information, and/or combinations thereof. In this manner, a surgeon may evaluate potential outcomes of the surgical procedure while performing the implantation of annuloplasty ring 660.

As shown in FIGS. 6B and 6D-6H, annuloplasty ring 660 may have a shape 670 formed based on anchors 640 (e.g., vertices) and struts between anchors (e.g., edges). In some embodiments, shape 670 may have a star or substantially star-like shape. Comparing shape 670 in FIG. 6D with shape 670 in FIG. 6H, adjustments to annuloplasty ring 660 and/or components thereof may create different shapes 670. In some embodiments, components of annuloplasty ring 660 (such as anchors 640 and/or collars 642) may be associated with coordinates, for example, relative to other components and/or patient anatomy (for instance, anchor 640-1 may have a coordinate of (−5 mm, 20 mm) with respect to a central point of valve 621, anchor 640-2 may have a coordinate of (15 mm, 15 mm), and so on).

As described in reference to FIG. 2, shape 670 and/or coordinates may be used to determine characteristics of the implantation of annuloplasty ring 660, such as comparison with an optimized configuration, violation of surgical guidelines or performance goals, and/or the like. For example, shape 670 and/or coordinates may be compared to an optimized configuration determined for the particular patient based on patient characteristics. During a procedure, a surgeon may view shape 670 and/or coordinates in comparison to an optimized shape and/or optimized coordinates (for instance, as an overlay on user interface 612) and/or may be presented with indicators to guide the surgeon to an optimized placement and/or configuration.

Accordingly, in some embodiments, user interface 612 may operate to provide a simulated (or partially simulated) visualization of annuloplasty ring 660 within the body of a patient. In some embodiments, a surgeon may emulate the physical placement and configuration of annuloplasty ring 660 by setting the configuration information of annuloplasty ring 660 to match the physical device within the patient. The view of annuloplasty ring 660 via user interface 612 may be improved compared with conventional views of implantable medical devices. For example, through user interface 612, a surgeon may see a clear, comprehensive top-down view of annuloplasty ring 660 arranged over a heart valve. For a non-invasive, catheter-based approach, such a view is a vast improvement over the use of manual data tracking (for instance, “whiteboarding”) and incomplete diagnostic images. Accordingly, emulation of annuloplasty ring 660 via user interface 612 may provide multiple advantages to a surgeon, including, without limitation, a less complicated surgical approach, more efficient surgical planning, improved device implantation, and a more accurate and comprehensive view of the surgical field with the annuloplasty ring device.

In some embodiments, the placement and/or configuration of the physical annuloplasty ring within the patient may correspond directly with annuloplasty ring 660 (which may be a simulated image or a real-time image). For example, if a surgeon adjusts a component of annuloplasty ring 660, such as moving anchor 640-1 from the leaflet to the atrium within the patient via manual manipulation of the deployment device, the image of annuloplasty ring 660 within user interface 612 may be updated to correspond to the physical position and configuration within the patient. For example, the computer-assisted surgical process (for instance, via deployment application 148) may receive sensor information associated with annuloplasty ring 660 and/or components thereof, interpret the sensor information to determine the position and/or configuration, and provide an updated visualization of annuloplasty ring 660 that corresponds with the physical device within the patient.

In some embodiments, manipulation of annuloplasty ring 660, such as rotating annuloplasty ring 660 clockwise 90 degrees via rotation object 690, may cause a corresponding physical rotation of the device within the patient. For example, manipulation of a graphical object on user interface 612 may cause a corresponding manipulation of the physical device within the patient. For instance, the computer-assisted surgical process (for instance, via deployment application 148) may receive virtual manipulation information resulting from manipulation of objects of user interface 612 (for example, anchors 640, slider 661, rotation object 690) and generate control signals that may be transmitted to annuloplasty ring 660 and/or deployment device 170 (for automatically manipulating annuloplasty ring 660).

In some embodiments, heart 620 and annuloplasty ring 660 may be a live image, for instance, captured via one or more camera and/or diagnostic image device (for instance, an ICE device). In various embodiments, certain features, such as anchors 640, collars 642, indicators 672 and 674, may be overlaid (along with value or implantation information, such as 644 and/or 646) onto the real-time image. For instance, the computer-assisted surgical process (for instance, via deployment application 148) may receive image information and determine the location of heart 620 and/or annuloplasty ring 660 objects in the video image. The object determinations may be via image analysis and/or user input indicate areas with objects. In this manner, device and/or anatomical objects along with metadata, such as anchor placement, adjustment values, indicators, may be presented in combination with real-time image information to provide an improved and more efficient surgical process via visualization of the heart 620 and annuloplasty ring 660 and related objects. Included herein are one or more logic flows, workflows, and/or combinations thereof representative of exemplary methodologies for performing novel aspects of the disclosed embodiments. While, for purposes of simplicity of explanation, the one or more methodologies shown herein are shown and described as a series of acts, those skilled in the art will understand and appreciate that the methodologies are not limited by the order of acts. Some acts may, in accordance therewith, occur in a different order and/or concurrently with other acts from that shown and described herein. For example, those skilled in the art will understand and appreciate that a methodology could alternatively be represented as a series of interrelated states or events, such as in a state diagram. Moreover, not all acts illustrated in a methodology may be required for a novel implementation.

A logic flow and/or workflow may be implemented in software, firmware, hardware, or any combination thereof and/or may be combined with manual steps. In software and firmware embodiments, a logic flow may be implemented by computer executable instructions stored on a non-transitory computer readable medium or machine readable medium. The embodiments are not limited in this context.

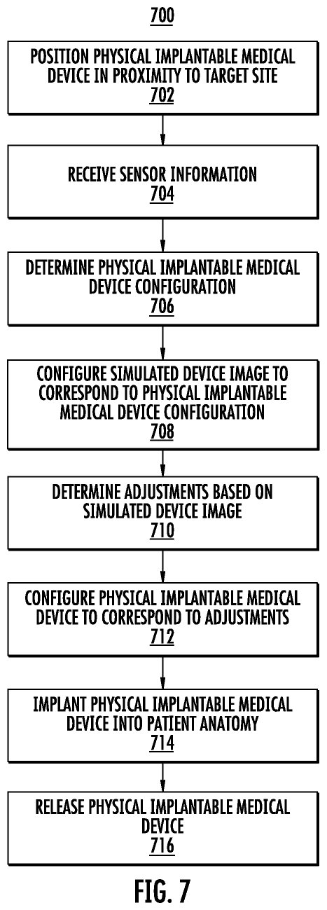

FIG. 7 illustrates an embodiment of a workflow 700. Workflow 700 may be representative of some or all of the operations performed according to one or more embodiments described herein, such as computer-assisted deployment system 105 and/or components thereof (for instance, computing device 110). In some embodiments, workflow 700 may be representative of some or all of the operations of a non-invasive transcatheter surgical process to implant an implantable medical device according to some embodiments.

At block 702, workflow 700 may include positioning a physical implantable medical device in proximity to a target site. For example, a surgeon may deliver an annuloplasty ring in proximity to a mitral valve within a heart of a patient. Delivery of the annuloplasty ring may include inserting an ICE catheter until the mitral valve/device is visible on a display device configured to present the ICE device images.

Workflow 700 may include receiving sensor information at block 704. For example, an annuloplasty ring may be associated with various sensors, such as imaging devices (for instance, an ICE device), position sensors, orientation sensors, accelerometers, and/or the like. In some embodiments, components of annuloplasty ring (such as anchors) may have position sensors so that at least a portion of the components may be able to determine a relative location (for instance, computer-assisted surgical processes may operate to determine a position of anchor 1 with respect to anchors 2-8, anchor 2 with respect to anchors 1 and 3-8, and so on). In some embodiments, sensor information may include ICE images.

At block 706, workflow 700 may include determining a configuration of the physical implantable medical device. For example, the ICE catheter may be rotated until the anchor located on the mitral curtain is located (if there are two, one may be selected). The anchors may be moved to determine the anchors within the image. For example, anchor 1 may be moved via a deployment device and the anchor in the image with a corresponding anchor will be anchor 1. At this stage, the collars should be in a default open configuration (for instance, not cinched).

Workflow 700 may include configuring the simulated device image to correspond to the physical implantable medical device configuration at block 708. For example, a simulated device image (see, for example, 260 of FIGS. 2 and/or 660 of FIGS. 6B and 6D-6H) may be generated on a user interface, for instance, overlaid on a heart valve. The simulated device image may be manipulated (for instance, rotated, moved, and/or the like) to be in a position that corresponds to the configuration of the delivered implantable medical device. For example, the entire annuloplasty ring may be moved and/or rotated. In another example, device components, such as anchors, may be moved (for instance, from a first anatomical region (leaflet) to a second anatomical region (annulus)). Accordingly, the simulated image is configured to match or otherwise correspond to the configuration of the physical implantable medical device.

Workflow 700 may include determining adjustments based on the simulated device image at block 710. For example, an operator may rotate through all of the anchors while noting, for example, their position 1) relative to the annulus (e.g., on leaflet, on annulus/desired point, or atrial/too far from annulus) and 2) the height each anchor is positioned off the annulus (e.g., touching, able to hit with reach anchor, unable to hit with reach anchor, and/or the like). An operator may review the simulated device image and determine what adjustments need to be made. Adjustments may involve manipulating functions, including, without limitation, slider adjustment, device rotation, anchor location, gimbaling, and/or the like.

At block 712, workflow 700 may include configuring the physical implantable medical device to correspond to the adjustments. For example, an operator may use graphical user interface functions and/or a deployment device to effectuate configuration changes to the physical implantation device.

At block 714, workflow 700 may include implanting the physical implantable medical device to the patient anatomy. For example, one or more of the anchors of an annuloplasty ring may be installed, placed, or otherwise rotated into the valve anatomy at their particular placement region. In another example, collars may be adjusted to cinch one or more anchors of the physical implantable medical device.

In some embodiments, one or more of blocks 708-712 may be repeated (before, after, or in combination with block 714) to achieve a desired configuration of the physical implantable medical device. For example, an ICE sweep may be performed to update the configuration information for the annuloplasty ring. In some embodiments, information associated with the physical implantable medical device may be recorded, such as mitral measurements, regurgitation information, and/or the like.

At block 716, workflow 700 may include releasing the physical implantable medical device. For example, the connections between the anchors, collars, and other components of the annuloplasty ring to the deployment device may be disconnected and the catheter device may be removed from the patient.

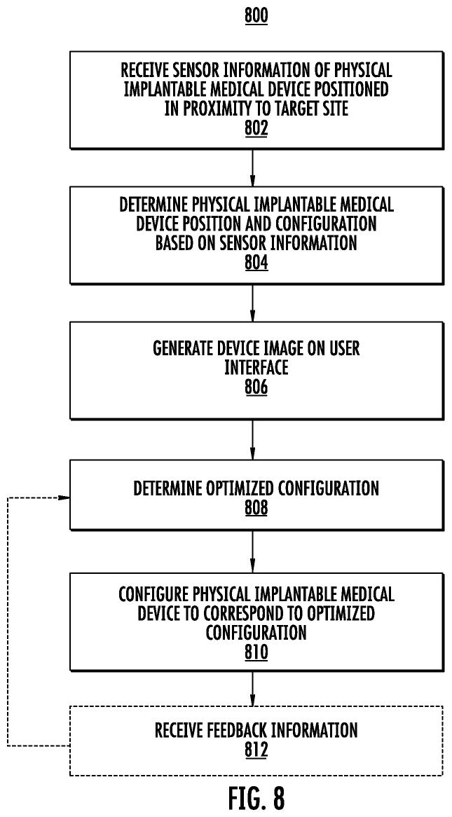

FIG. 8 illustrates an embodiment of a logic flow 800. Logic flow 800 may be representative of some or all of the operations executed by one or more embodiments described herein, such as surgery system 105 and/or components thereof (for instance, computing device 110). In some embodiments, logic flow 800 may be representative of some or all of the operations of a computer-assisted surgical process.

At block 802, logic flow 800 may receive sensor information associated with a physical implantable medical device delivered in proximity to a target site. For example, an annuloplasty ring may be associated with various sensors, such as imaging devices (for instance, an ICE device), position sensors, orientation sensors, accelerometers, and/or the like. A deployment application (for instance, deployment application 148) may receive the sensor information from the annuloplasty ring and/or associated deployment device.

Logic flow 800 may determine a position and configuration of the physical implantable medical device based on the sensor information at block 804. For example, a deployment application may operate to analyze the sensor information to determine the position of the annuloplasty ring within the patient anatomy and/or the positions/configurations of device components, such as anchors, collars, and/or the like.

At block 806, logic flow 800 may generate a device image on a user interface. For example, the deployment application may generate an image associated with the annuloplasty ring on a user interface (for example, user interface 212 and/or 612). In some embodiments, the device image may be or may include simulated images, objects, and/or the like. In various embodiments, the device image may include real-time images captured via a camera or diagnostic imaging system.

Logic flow 800 may determine an optimized configuration at block 808. For example, deployment application may access model information to determine an optimized configuration for the annuloplasty ring within the specific anatomy of the patient. In some embodiments, the optimized configuration may be generated as output of a trained ML model, such as a convoluted NN (CNN) and/or the like. In various embodiments, the optimized configuration may be or may include a shape and/or coordinates associated with the components of the annuloplasty ring, such as the anchors and the struts connecting the anchors. In exemplary embodiments, the optimized configuration may include parameter settings, such as slider adjustment values, anchor depth, and/or the like.

At block 810, logic flow 800 may configure the physical implantable medical device to correspond to the optimized configuration. In some embodiments, configuration of the physical annuloplasty ring may be via manual manipulation by a surgeon, such as through a deployment device. For example, a surgeon may manipulate a deployment device to configure the annuloplasty ring and/or components thereof to correspond to the optimized configuration. In various embodiments, one or more configuration steps may be performed automatically via motors, valves, wires, and/or other control elements coupled to the annuloplasty ring and/or components thereof. For example, the deployment application may determine adjustments to the annuloplasty ring and/or components thereof in order to achieve conformance with the optimized configuration. The deployment application may transmit control signals to the annuloplasty ring and/or deployment device to implement the adjustments.

Logic flow 800 may include an optimal step of receiving feedback information at block 812. For example, the deployment application may receive performance information, such as regurgitation information, heart performance information, patient vitals, surgeon-provided information (for instance, indicating a positive/negative outcome of a configuration), and/or the like. In some embodiments, the feedback information may be used to determine an optimized configuration, for instance, as part of a feedback loop for optimizing the placement and/or configuration of the annuloplasty ring and/or components thereof (for instance, placement of anchors, cinching of collars, and/or the like) in the current surgery and/or future surgeries (for instance, as part of model information to train computational models).

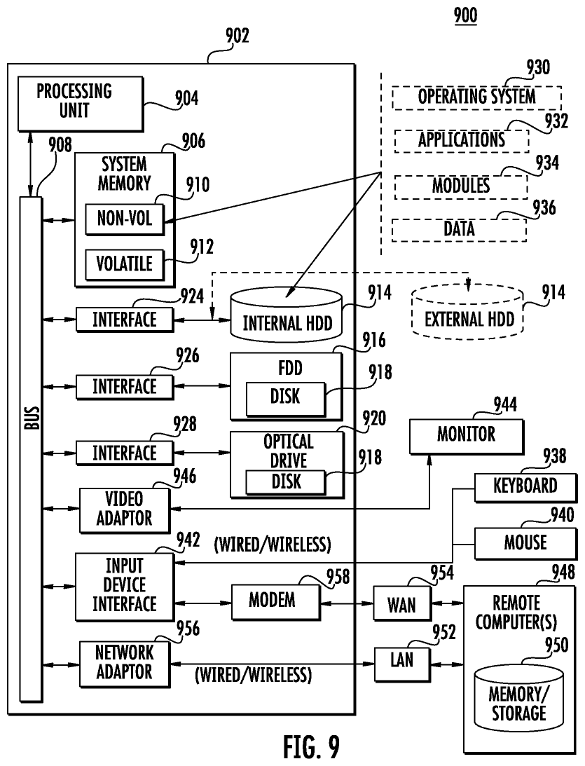

FIG. 9 illustrates an embodiment of an exemplary computing architecture 900 suitable for implementing various embodiments as previously described. In various embodiments, the computing architecture 900 may comprise or be implemented as part of an electronic device. In some embodiments, the computing architecture 900 may be representative, for example, of computing device 110. Embodiments are not limited in this context.

As used in this application, the terms “system, “component,” module,” and “logic” are intended to refer to a computer-related entity, either hardware, a combination of hardware and software, software, or software in execution, examples of which are provided by the exemplary computing architecture 900. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 900 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 900.

As shown in FIG. 9, the computing architecture 900 comprises a processing unit 904, a system memory 906 and a system bus 908. The processing unit 904 may be a commercially available processor and may include dual microprocessors, multi-core processors, and other multi-processor architectures.

The system bus 908 provides an interface for system components including, but not limited to, the system memory 906 to the processing unit 904. The system bus 908 can be any of several types of bus structure that may further interconnect to a memory bus (with or without a memory controller), a peripheral bus, and a local bus using any of a variety of commercially available bus architectures. Interface adapters may connect to the system bus 908 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 906 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 9, the system memory 906 can include non-volatile memory 910 and/or volatile memory 912. A basic input/output system (BIOS) can be stored in the non-volatile memory 910.

The computer 902 may include various types of computer-readable storage media in the form of one or more lower speed memory units, including an internal (or external) hard disk drive (HDD) 914, a magnetic floppy disk drive (FDD) 916 to read from or write to a removable magnetic disk 911, and an optical disk drive 920 to read from or write to a removable optical disk 922 (e.g., a CD-ROM or DVD). The HDD 914, FDD 916 and optical disk drive 920 can be connected to the system bus 908 by an HDD interface 924, an FDD interface 926 and an optical drive interface 928, respectively. The HDD interface 924 for external drive implementations can include at least one or both of Universal Serial Bus (USB) and IEEE 1114 interface technologies.

The drives and associated computer-readable media provide volatile and/or nonvolatile storage of data, data structures, computer-executable instructions, and so forth. For example, a number of program modules can be stored in the drives and memory units 910, 912, including an operating system 930, one or more application programs 932, other program modules 934, and program data 936. In one embodiment, the one or more application programs 932, other program modules 934, and program data 936 can include, for example, the various applications and/or components of computing device 110.

A user can enter commands and information into the computer 902 through one or more wired/wireless input devices, for example, a keyboard 938 and a pointing device, such as a mouse 940. These and other input devices are often connected to the processing unit 904 through an input device interface 942 that is coupled to the system bus 908, but can be connected by other interfaces.

A monitor 944 or other type of display device is also connected to the system bus 908 via an interface, such as a video adaptor 946. The monitor 944 may be internal or external to the computer 902. In addition to the monitor 944, a computer typically includes other peripheral output devices, such as speakers, printers, and so forth.

The computer 902 may operate in a networked environment using logical connections via wired and/or wireless communications to one or more remote computers, such as a remote computer 948. The remote computer 948 can be a workstation, a server computer, a router, a personal computer, portable computer, microprocessor-based entertainment appliance, a peer device or other common network node, and typically includes many or all of the elements described relative to the computer 902, although, for purposes of brevity, only a memory/storage device 950 is illustrated. The logical connections depicted include wired/wireless connectivity to a local area network (LAN) 952 and/or larger networks, for example, a wide area network (WAN) 954. Such LAN and WAN networking environments are commonplace in offices and companies, and facilitate enterprise-wide computer networks, such as intranets, all of which may connect to a global communications network, for example, the Internet.

The computer 902 is operable to communicate with wired and wireless devices or entities using the IEEE 802 family of standards, such as wireless devices operatively disposed in wireless communication (e.g., IEEE 802.16 over-the-air modulation techniques). This includes at least Wi-Fi (or Wireless Fidelity), WiMAX, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The following claims are hereby incorporated into this Detailed Description by this reference, with each claim standing on its own as a separate embodiment of the present disclosure. Reference signs in the claims are provided merely as a clarifying example and shall not be construed as limiting the scope of the claims in any way.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “the”, “first”, “second”, etc., do not preclude a plurality. Reference signs in the claims are provided merely as a clarifying example and shall not be construed as limiting the scope of the claims in any way.

What is claimed is:

1. A method of deploying an annuloplasty ring within a heart of a patient for a closed valve repair procedure, the method comprising:
positioning the annuloplasty ring adjacent to a valve within a heart of the patient, the annuloplasty ring comprising a plurality of anchors, a plurality of collars, and at least one sensor configured to provide sensor information indicating one of a position or configuration of the annuloplasty ring;
receiving, at a logic device, sensor information associated with the annuloplasty ring;
determine configuration information of the annuloplasty ring within the heart of the patient;
displaying, via the logic device, a device image corresponding to the annuloplasty ring based on the configuration information;
receiving input, at the logic device, configuring the device image to correspond to the configuration information;
determining at least one adjustment for the annuloplasty ring based on the device image;
configuring the annuloplasty ring based on the at least one adjustment; and
implant the annuloplasty ring within the patient by threading the anchors into valve tissue.

2. The method of claim 1, the at least one sensor comprising at least one of a camera imaging device or a diagnostic imaging device.

3. The method of claim 1, the at least one sensor comprising an intravascular cardiac echography (ICE) catheter arranged within the annuloplasty ring.

4. The method of claim 1, the configuration information comprising at least one of an anchor placement status of the plurality of anchors or an adjustment value of the plurality of collars.

5. The method of claim 1, the device image comprising a simulated image overlaid on an image of a valve portion of the heart of the patient.

6. The method of claim 1, the at least one adjustment comprising at least one of placement of the annuloplasty ring, placement of at least one of the plurality of anchors, or adjustment of at least one of the plurality of collars.

7. The method of claim 1, comprising determining an optimized configuration of the annuloplasty ring based on at least one of a shape of a frame of the annuloplasty ring or coordinates of the anchors.

8. The method of claim 1, comprising, via the logic device, displaying at least one adjustment indicator to indicate an adjustment of at least one of the annuloplasty ring or at least one of the plurality of anchors.

9. The method of claim 1, the at least one adjustment based on a determination that placement of at least one of the plurality of anchors will not enter sufficiently anchor into the valve tissue.

10. The method of claim 1, the configuration of the annuloplasty ring comprising a configuration of the plurality of anchors and the plurality of collars.

11. An apparatus, comprising:
a storage device; and
logic, at least a portion of the logic implemented in circuitry coupled to the storage device to implement an implantation process to deploy an implantable medical device within a patient via a closed surgical process, the logic to:
receive sensor information from at least one sensor associated with the implantable medical device, the implantable medical device comprising a plurality of components,
determine position information and configuration information of the implantable medical device based on the sensor information,
generate a device image corresponding to the implantable medical device based on the position information and the configuration information, and
determine an optimized configuration based on at least one configuration characteristic of the plurality of components using at least one computational model, the at least one configuration characteristic comprising one of a shape of the implantable medical device or component coordinates.

12. The apparatus of claim 11, the implantable medical device comprising an annuloplasty ring.

13. The apparatus of claim 11, the at least one sensor comprising at least one of a camera imaging device, a diagnostic imaging device, a position sensor, or an accelerometer sensor.

14. The apparatus of claim 11, the position information comprising a position of the implantable medical device in relation to a target site and the configuration information comprising at least one configuration setting of at least one of the plurality of components.

15. The apparatus of claim 11, the logic operative to cause an adjustment of at least one of the plurality of components to conform the implantable medical device to the optimized configuration.

16. A computer-implemented method to deploy an implantable medical device within a patient via a closed surgical process, the method comprising, via a computing device operably coupled to the implantable medical device:
receiving sensor information from at least one sensor associated with the implantable medical device, the implantable medical device comprising a plurality of components;
determining position information and configuration information of the implantable medical device based on the sensor information;
generating a device image corresponding to the implantable medical device based on the position information and the configuration information; and
determining an optimized configuration based on at least one configuration characteristic of the plurality of components using at least one computational model, the at least one configuration characteristic comprising one of a shape of the implantable medical device or component coordinates.

17. The computer-implemented method of claim 11, the implantable medical device comprising an annuloplasty ring.

18. The computer-implemented method of claim 11, the at least one sensor comprising at least one of a camera imaging device, a diagnostic imaging device, a position sensor, or an accelerometer sensor.

19. The computer-implemented method of claim 11, the position information comprising a position of the implantable medical device in relation to a target site and the configuration information comprising at least one configuration setting of at least one of the plurality of components.

20. The computer-implemented method of claim 11, further comprising causing an adjustment of at least one of the plurality of components to conform the implantable medical device to the optimized configuration.
